(19) United States
(12) Patent Application Publication
(10) Pub. No.: US 2019/0017017 A1
(43) Pub. Date: Jan. 17, 2019

(54) USE OF VASCULAR CELLS TO CREATE THE CONVENTIONAL OUTFLOW TRACT

(71) Applicant: THE RESEARCH FOUNDATION FOR THE STATE UNIVERSITY OF NEW YORK, Albany, NY (US)

(72) Inventors: Yubing Xie, Cohoes, NY (US); Karen Torrejon, Ballston Spa, NY (US); Susan Sharfstein, Niskayuna, NY (US); Magnus Bergkvist, Albany, NY (US); John Danias, Staten Island, NY (US); Cula N. Dautriche, Brooklyn, NY (US)

(21) Appl. No.: 16/044,806

(22) Filed: Jul. 25, 2018

Related U.S. Application Data
(63) Continuation of application No. PCT/US2017/014892, filed on Jan. 25, 2017.
Application No. PCT/US2017/014892 is a continuation of application No. PCT/IB2017/051424, filed on Mar. 10, 2017.
(60) Provisional application No. 62/286,760, filed on Jan. 25, 2016.
(60) Provisional application No. 62/286,743, filed on Jan. 25, 2016.
(60) Provisional application No. 62/286,760, filed on Jan. 25, 2016.
(60) Provisional application No. 62/286,743, filed on Jan. 25, 2016.

(51) Int. Cl. C12N 5/079; C12M 1/12; C12M 1/42

(52) U.S. Cl. CPC C12N 5/0621; C12M 25/14; C12M 35/08; C12N 2513/00; C12N 2533/32; C12N 2503/02; C12N 2533/52; C12N 2533/54

(57) ABSTRACT

Provided is a system for modeling the conventional outflow tract, including: a porous scaffold with trabecular meshwork cells attached to one surface and microvascular endothelial cells co-cultured on an opposite surface transforming the microvascular endothelial cells into Schlemm's canal cell-like cells. Also provided is a method for using the system for screening by contacting the cells with a known or suspected medicament and measuring its effects on the system such as flow of a perfusate. Also provided is a method of making the system by fabricating the porous substrate as a micropatterned scaffold.

### CROSS REFERENCE TO RELATED APPLICATIONS

This application is a continuation of International Application No. PCT/US2017/014892, filed Jan. 25, 2017, and International Application No. PCT/IB2017/051424, filed Mar. 10, 2017, both of which claim priority to U.S. Provisional Patent Application No. 62/286,760, filed on Jan. 25, 2016, and to U.S. Provisional Patent Application No. 62/286,743, filed on Jan. 25, 2016, the entire disclosure of these applications being hereby incorporated herein by reference.

### GOVERNMENT RIGHTS STATEMENT

This invention was made with Government support under grant EY020670 awarded by the National Institutes of Health and STTR grant 448900 awarded by the National Science Foundation. The U.S. Government has certain rights in the invention.

### SEQUENCE LISTING

This application contains a Sequence Listing, created on Jan. 20, 2016; the file, in ASCII format, is designated 2835151AWO_SEQ.txt and is 2.3 kilobytes in size. The file is hereby incorporated by reference in its entirety into the present application.

### TECHNICAL FIELD

The present disclosure generally relates to a system for modeling the conventional outflow track of the mammalian eye. More particularly, the present disclosure relates to transforming microvascular cells into Schlemm's canal cell-like cells by co-culturing them with trabecular meshwork cells on a scaffold to mimic the biology and physiology of conventional outflow track function.

### BACKGROUND OF THE INVENTION

Glaucoma is the leading cause of irreversible blindness, resulting from an increase in intraocular pressure (IOP). IOP is the only modifiable risk factor of glaucoma and is controlled by the outflow of the aqueous humor through the conventional outflow track of the eye. The conventional outflow tract is populated by two cell types: cells of the trabecular meshwork (TM) and cells of Schlemm's canal (SC). Before entering the venous system, the aqueous humor (AH) has to pass through the TM and then enter the inner wall of the SC. Together, these structures are responsible for providing resistance to the outflow of the AH and maintain intraocular pressure (IOP) in the eye.

Cells of the TM and SC have features and responsibilities that contribute to the overall generation and regulation of outflow resistance in both healthy and diseased conventional outflow tract. Isolation of human trabecular meshwork (HTM) cells is now a fairly common technique but human Schlemm's canal (HSC) cell isolation is uncommon, not readily reproduced, or of generally limited success. Thus, to better model conventional outflow tract function to treat disease states in which its malfunction is implicated, there is a need for a more reproducible, reliable source of cells that can serve as a proxy for SC cells. Furthermore, long-term, properly targeted transgene expression or gene knockdown in the trabecular meshwork (TM) and Schlemm's canal (SC) cells which constitute the conventional outflow tract has not been achieved by conventional methodology. Methods and systems are needed that allow for transfection of TM and SC cells and models thereof.

### SUMMARY OF THE INVENTION

The shortcomings of the prior art are overcome, and additional advantages are provided, through the provision, in one aspect, of a system for modeling the conventional outflow tract, including: a porous scaffold with a first surface on a side opposite a second surface; a plurality of trabecular meshwork cells attached to the first surface and extending into a plurality of pores in the porous scaffold; and a plurality of second cells attached to the second surface and extending into the plurality of pores, wherein the second cells include microvascular endothelial cells that were co-cultured on the scaffold with the plurality of trabecular meshwork cells for a period of time to form Schlemm's canal cell-like cells, Schlemm's canal cells, or both. The porous scaffold may be a micropatterned substrate, which may include a photoresist, a thermoplastic polymer, or a thermoset polymer.

In some embodiments, the porous scaffold may include a coating. As non-limiting examples, the coating may include poly-L-lysine, gelatin, a hyaluronic acid-based polymer, a hydrogel, a hyaluronic acid-based hydrogel, collagen, fibronectin, vitronectin, chitosan, extracellular matrix, an RGD-containing peptide, a peptide attached to acrylate, or any combination of two or more of the foregoing.

In some embodiments, the porous scaffold may have a pore width of between 200 nm and 1 μm, between 1 μm and 5 μm, between 5 μm, and 10 μm, between 10 μm and 15 μm, between 15 μm and 20 μm, between 11 μm and 13 μm, between 3 μm and 9 μm, between 7 μm and 8 μm, or between 7 μm and 15 μm. In other embodiments, the porous scaffold may have a thickness of less than 200 nm, between 200 nm to 1 μm, between 1 μm and 40 μm, or a thickness of 20 μm.

In some examples, the period of time may be seven days or longer. The plurality of trabecular meshwork cells and the plurality of second cells may also contact each other through a plurality of pores in the porous scaffold. In further embodiments, the plurality of trabecular meshwork cells, the second cells, or both, are transfected or are contacted with a known or suspected medicament or a compound known or suspected to impair, enhance, ameliorate, or improve conventional outflow tract function.

In some examples, a micropatterned substrate may be SU-8, a coating may be gelatin, a pore width may be between 11 μm and 13 μm, and a scaffold thickness may be 20 μm, the plurality of trabecular meshwork cells and the plurality of second cells may contact each other through a plurality of pores in the porous scaffold, the period of time is seven days or longer, and the plurality of trabecular meshwork cells, the plurality of second cells, or both, are transfected or are contacted with a known or suspected medicament or a compound known or suspected to impair, enhance, ameliorate, or improve conventional outflow tract function.

Also provided is a method for screening including obtaining a porous scaffold, that includes a first surface on a side opposite a second surface; a plurality of trabecular meshwork cells on the first surface wherein the plurality of trabecular meshwork cells were formed by seeding and growing a plurality of trabecular meshwork cells on the first surface; a plurality of second cells seeded and grown on the second surface, wherein the second cells include microvascular endothelial cells that were co-cultured with the plurality of trabecular meshwork cells on the first surface for a period of time at least until the plurality of microvascular endothelial cells were transformed into a plurality of Schlemm's canal cell-like cells, Schlemm's canal cells, or both; contacting the plurality of trabecular meshwork cells, the plurality of second cells, or both, with a known or suspected medicament or a compound known or suspected to impair, enhance, ameliorate, or improve conventional outflow tract function, or transfecting the plurality of trabecular meshwork cells, the plurality of second cells, or both; perfusing fluid through the plurality of trabecular meshwork cells and the plurality of second cells; and measuring the transmembrane pressure, flow rate, through-flow, resistance to flow, hydraulic conductivity, vacuole dynamics, pore formation, or outflow facility of the plurality of trabecular meshwork cells and the plurality of second cells.

In some embodiments, the porous scaffold may include a coating. As non-limiting examples, the coating may include poly-L-lysine, gelatin, a hyaluronic acid-based polymer, a hydrogel, a hyaluronic acid-based hydrogel, collagen, fibronectin, vitronectin, chitosan, extracellular matrix, an RGD-containing peptide, a peptide attached to acrylate, and any combination of two or more of the foregoing.

In some embodiments porous scaffold may have a pore width of between 200 nm and 1 μm, between 1 μm and 5 μm, between 5 μm, and 10 μm, between 10 μm and 15 μm, between 15 μm and 20 μm, between 11 μm and 13 μm, between 3 μm and 9 μm, between 7 μm and 8 μm, or between 7 μm and 15 μm. In other embodiments, the porous scaffold may have a thickness of less than 200 nm, between 200 nm to 1 μm, between 1 μm and 40 μm, or a thickness of 20 μm.

In certain embodiments, seeding and growing a plurality of trabecular meshwork cells may include seeding an initial density of trabecular meshwork cells of greater than 10,000 cells/cm². In an example, the initial density of trabecular meshwork cells is at least 40,000 cells/cm². In yet other embodiments, the second cells may be seeded at an initial density of greater than 10,000 cells/cm². In an example, the second cells may be seeded at an initial density of at least 40,000 cells/cm². In yet another embodiments, the period of time may be seven days or longer. In still another embodiment, the plurality of trabecular meshwork cells and the plurality of second cells may also contact each other through a plurality of pores in the porous scaffold.

In some examples, a micropatterned substrate may be SU-8, a coating may be gelatin, a pore width may be between 11 μm and 13 μm, and a scaffold thickness may be 20 μm, the plurality of trabecular meshwork cells and the plurality of second cells may contact each other through a plurality of pores in the porous scaffold, the period of time is seven days or longer.

Also provided is a method of making a model of a conventional outflow tract, including obtaining a porous scaffold that includes a first surface on a side opposite a second surface; seeding and growing a plurality of trabecular meshwork cells on the first surface; and seeding and growing a plurality of second cells on the second surface wherein the second cells comprise Schlemm's canal cells or are microvascular endothelial cells and co-culturing the plurality of trabecular meshwork cells on the first surface for a period of time at least until the plurality of microvascular endothelial cells are transformed into a plurality of Schlemm's canal cell-like cells. In an embodiment, the method of making a model of a conventional outflow tract may also include a porous scaffold that is a micropatterned substrate. In some examples, the micropatterned substrate may include a photoresist, a thermoset polymer, or a thermoplastic polymer. Another embodiment may include forming the micropatterned substrate by depositing a photoresist layer on a surface, masking the photoresist layer with a photomask, and creating pores by irradiating portions of the photoresist layer not masked by the photomask and developing the photoresist layer, and releasing the photoresist layer from the surface.

In yet other embodiments, the photoresist layer may be SU-8. Still further embodiments may include depositing a coating on the micropatterned substrate. As non-limiting examples, the coating may include poly-L-lysine, gelatin, a hyaluronic acid-based polymer, a hydrogel, a hyaluronic acid-based hydrogel, collagen, fibronectin, vitronectin, chitosan, extracellular matrix, an RGD-containing peptide, a peptide attached to acrylate, and any combination of two or more of the foregoing.

In some embodiments the porous scaffold may have a pore width of between 200 nm and 1 μm, between 1 μm and 5 μm, between 5 μm, and 10 μm, between 10 μm and 15 μm, between 15 μm and 20 μm, between 11 μm and 13 μm, between 3 μm and 9 μm, between 7 μm and 8 μm, or between 7 μm and 15 μm. In other embodiments, the porous scaffold may have a thickness of less than 200 nm, between 200 nm to 1 μm, between 1 μm and 40 μm, or a thickness of 20 μm.

In certain embodiments, seeding and growing a plurality of trabecular meshwork cells may include seeding an initial density of trabecular meshwork cells of greater than 10,000 cells/cm². In an example, the initial density of trabecular meshwork cells is at least 40,000 cells/cm². In yet other embodiments, the second cells may be seeded at an initial density of greater than 10,000 cells/cm². In an example, the second cells may be seeded at an initial density of at least 40,000 cells/cm². In yet another embodiments, the period of time may be seven days or longer. In still another embodiment, the plurality of trabecular meshwork cells and the plurality of second cells may also contact each other through a plurality of pores in the porous scaffold.

In certain embodiments, seeding and growing a plurality of trabecular meshwork cells may include seeding an initial density of trabecular meshwork cells of greater than 10,000 cells/cm². In an example, the initial density of trabecular meshwork cells is at least 40,000 cells/cm². In yet other embodiments, the second cells may be seeded at an initial density of greater than 10,000 cells/cm². In an example, the second cells may be seeded at an initial density of at least 40,000 cells/cm². In yet another embodiments, the period of time may be seven days or longer. In still another embodiment, the plurality of trabecular meshwork cells and the plurality of second cells may also contact each other through a plurality of pores in the porous scaffold.

In still another aspect, provided is a system for modeling the conventional outflow tract, including: a porous scaffold with a first surface on a side opposite a second surface; a plurality of first cells attached to the first surface and extending into a plurality of pores in the porous scaffold, wherein the first cells comprise trabecular meshwork cells, stem cells that can differentiate into trabecular meshwork cells, precursor trabecular meshwork cells, or any combination of two or more of the foregoing; and a plurality of second cells attached to the second surface and extending into the plurality of pores, wherein the second cells comprise microvascular endothelial cells that were co-cultured on the scaffold with the plurality of first cells for a period of time to form Schlemm's canal cell-like cells, stem cells that can be differentiated into vascular endothelial cells that were co-cultured on the scaffold with the plurality of first cells for a period of time to form Schlemm's canal cell-like cells, precursor vascular endothelial cells that were co-cultured on the scaffold with the plurality of first cells for a period of time to form Schlemm's canal cell-like cells, stem cells that can be differentiated to Schlemm's canal cells that were co-cultured on the scaffold with the plurality of first cells for a period of time to form Schlemm's canal cell-like cells, Schlemm's canal cells, or any combination of two or more of the foregoing. The porous scaffold may be a micropatterned substrate, which may include a photoresist, a thermoplastic polymer, or a thermoset polymer.

In still another aspect, provided is a method for screening including obtaining a porous scaffold, that includes a first surface on a side opposite a second surface; a plurality of first cells on the first surface, wherein the first cells include trabecular meshwork cells, stem cells that can differentiate into trabecular meshwork cells, precursor trabecular meshwork cells, or any combination of two or more of the foregoing; and a plurality second cells on the second surface, wherein the second cells include microvascular endothelial cells that were co-cultured on the scaffold with the plurality of first cells for a period of time to form Schlemm's canal cell-like cells, stem cells that can differentiate into vascular endothelial cells that were co-cultured on the scaffold with the plurality of first cells for a period of time to form Schlemm's canal cell-like cells, precursor vascular endothelial cells that were co-cultured on the scaffold with the plurality of first cells for a period of time to form Schlemm's canal cell-like cells, stem cells that can be differentiated into Schlemm's canal cells that were co-cultured on the scaffold with the plurality of first cells for a period of time to form Schlemm's canal cell-like cells, Schlemm's canal cells, or any combination of two or more of the foregoing; contacting the plurality of first cells, the plurality of second cells, or both, with a known or suspected medicament or a compound known or suspected to impair, enhance, ameliorate, or improve conventional outflow tract function, or transfecting the plurality of first cells, the plurality of second cells, or both; perfusing fluid through the plurality of first cells and the plurality of second cells; and measuring the transmembrane pressure, flow rate, through-flow, resistance to flow, hydraulic conductivity, vacuole dynamics, pore formation, or outflow facility of the plurality of first cells and the plurality of second cells.

In yet a further aspect, provided is a method of making a model of a conventional outflow tract, which method includes obtaining a porous scaffold wherein the porous scaffold includes a first surface and a second surface and the first surface is on a side opposite the second surface; seeding and growing a plurality of first cells on the first surface, wherein the first cells include trabecular meshwork cells, stem cells that can differentiate into trabecular meshwork cells, precursor trabecular meshwork cells, or any combination of two or more of the foregoing; and seeding and growing a plurality of second cells on the second surface wherein the second cells comprise microvascular endothelial cells that form Schlemm's canal cell-like cells when co-cultured on the scaffold with the plurality of first cells for a period of time, stem cells that can differentiate into vascular endothelial cells that form Schlemm's canal cell-like cells when co-cultured on the scaffold with the plurality of first cells for a period of time, precursor vascular endothelial cells that form Schlemm's canal cell-like cells when co-cultured on the scaffold with the plurality of first cells for a period of time to form Schlemm's canal cell-like cells, stem cells that can be differentiated to Schlemm's canal cells that were co-cultured on the scaffold with the plurality of first cells for a period of time to form Schlemm's canal cell-like cells, Schlemm's canal cells, or any combination of two or more of the foregoing.

Additional features and advantages are realized through the techniques of the present disclosure. These and other objects, features and advantages of aspects of the present disclosure will become apparent from the following detailed description thereof taken in conjunction with the accompanying drawings.

### BRIEF DESCRIPTION OF THE DRAWINGS

One or more aspects of the present disclosure are particularly pointed out and distinctly claimed as examples in the claims at the conclusion of the specification. The foregoing and other objects, features, and advantages of the present disclosure are apparent from the following detailed description taken in conjunction with the accompanying drawings in which:

FIGS. 1A, 1B, 1C, 1D, 1E, 1F, and 1G are illustrations of a process for manufacturing a porous scaffold and a scaffold manufactured by the illustrated process;

FIGS. 2A, 2B, and 2C are scanning electron micrographs of different cell types on a porous scaffold;

FIGS. 3A and 3B are photomicrographs of microvascular cells grown on a microporous scaffold;

FIGS. 4A and 4B are comparisons between the expression patterns of proteins between microvascular endothelial cells and Schlemm's canal cells;

FIGS. 5A, 5B, 5C, and 5D are scanning electron micrographs of microvascular endothelial cells cultured in different media;

FIGS. 6A, 6B, 6C, and 6D are confocal micrographic images of microvascular endothelial cells cultured on porous scaffolds with different media for 7 days;

FIGS. 7A, 7B, 7C, and 7D are confocal micrographic images of microvascular endothelial cells cultured on porous scaffolds with different media for 14 days;

FIGS. 8A, 8B, 8C, 8D, 8E, and 8F are scanning electron micrographs of microvascular endothelial cells co-cultured with trabecular meshwork cells on a porous scaffold for 7 and 14 days;

FIG. 9A is a diagrammatic representation of a protocol for co-culturing TM and microvascular endothelial cells on a porous scaffold;

FIGS. 9B and 9C are SEM and confocal micrographs, respectively, of TM and microvascular endothelial cells that have been co-cultured on a porous scaffold;

FIGS. 10A, 10B, 10C, and 10D are confocal images showing expression patterns of proteins in TM cells grown on a porous scaffold;

FIGS. 11A, 11B, 11C, and 11D are SEM images of SC and microvascular endothelial cells co-cultured with TM cells for 14 days;

FIGS. 12A, 12B, 12C, and 12D show comparisons of protein expression patterns of SC or microvascular endothelial cells co-cultured with TM cells on a porous scaffold for 14 days;

FIG. 13A is a diagram of a flow system used for perfusion studies;

FIG. 13B is a graph showing trans-tissue pressure of microvascular endothelial cells and SC cells co-cultured with TM cells;

FIGS. 14A, 14B, 14C, 14D, and 14E are qPCR and western blot analyses of outflow pathway marker expression in microvascular endothelial cells and SC cells co-cultured with TM cells and exposed to pharmacologic agents that regulate outflow;

FIG. 15 is a graph showing outflow facility of TM cells grown on a porous scaffold and treated with pharmacological agents known to modulate outflow facility;

FIG. 16A is confocal images of TM cells, and microvascular endothelial or SC cells co-cultured with TM cells on a porous scaffold transfected by an siRNA;

FIG. 16B is a graph showing the effect on outflow facility of transfecting TM cells grown on a porous scaffold with siRNAs that knock down SPARC, RhoA, and VEGF expression;

FIGS. 17A and 17B show the effectiveness and effects of siRNA transfection of HTM cells grown on a scaffold in accordance with aspects of the present disclosure; and

FIGS. 18A, 18B, 18C, and 18D show the effectiveness and effects of siRNA transfection of SC cells grown on a scaffold in accordance with aspects of the present disclosure.

### DETAILED DESCRIPTION OF THE INVENTION

Aspects of the present disclosure and certain features, advantages, and details thereof, are explained more fully below with reference to the non-limiting embodiments illustrated in the accompanying drawings. Descriptions of well-known materials, fabrication tools, processing techniques, etc., are omitted so as to not unnecessarily obscure the aspects of the present disclosure in detail. It should be understood, however, that the detailed description and the specific examples, while indicating embodiments of the disclosure, are given by way of illustration only, and are not by way of limitation. Various substitutions, modifications, additions and/or arrangements within the spirit and/or scope of the underlying inventive concepts will be apparent to those skilled in the art from this disclosure.

The present disclosure provides, in part, a system for modeling conventional outflow tract function. In one aspect, microvascular endothelial (MVE) cells are co-cultured with TM cells for a period of time on a porous scaffold until the MVE cells begin to exhibit morphological or biochemical signature characteristic of SC cells, whereupon they are referred to as SC cell-like cells. In another aspect, a method for screening is provided. SC cell-like cells and TM cells on a porous scaffold may be exposed to pharmacological agents and/or methods to manipulate gene expression and the effects of such treatments on function, such as those related to outflow facility, may be measured. In yet another aspect, a method of manufacturing a system for modeling conventional outflow tract function is provided. TM and MVE cells may be co-cultured on a porous scaffold for a period of time until the MVE cells become SC cell-like cells. In a further aspect, a porous scaffold on which MVE and TM cells are seeded may be manufactured by photolithographically processing a photoresist. Cells from any mammalian source of interest may be used, including but not limited to human, non-human primate, pig, horse, sheep, rodent, dog, cat, rabbit, and cow. It would be understood by skilled artisans that cells other than MVE cells and TM cells could also be used in placed of MVE cells and TM cells, respectively, in accordance with the present disclosure. For example, in an embodiment, stem cells with a TM cell phenotype or precursors of TM cells, or stem cells with an MVE phenotype MVE cell precursors, may be used in place of TM cells or MVE cells, respectively. Vascular endothelial cells other than microvascular endothelial cells, and stem cells or precursor cells for such vascular endothelial cells, could also be used in place of microvascular endothelial cells.

It would be appreciated by skilled artisans that stem cells that can differentiate into TM cells or precursor TM cells may be used in place of TM cells in accordance with aspects of the present invention. It would similarly be understood that stem cells that can differentiate into vascular endothelial cells, microvascular endothelial cells, or SC cells, or precursor vascular endothelial cells, microvascular endothelial cells, or SC cells could be used in place of microvascular endothelial cells or SC cells in accordance with aspects of the present disclosure. Furthermore, it would be understood that TM, SC, MVE, or vascular cells as those terms are used herein would be understood to include TM, SC, MVE, or vascular cells that are stem cells that differentiated into such cells, or are descendant cells of stem cells that differentiated into such cells types. Methods, systems, components, compositions, reagents, and other aspects described herein could be adapted by skilled artisans for use of the foregoing stem cells and/or precursor cells in accordance with well-known standard methodology, in keeping within aspects of the present disclosure. All such examples and permutations thereof are explicitly included as part of the present disclosure.

In one aspect, a porous scaffold is provided. A porous scaffold may be manufactured of any suitable material on which cells may adhere and grow. Examples include thermoplastic polymers, thermoset polymers, or photoresists that have been subjected to photolithographic processing. A thermoplastic or thermoset polymer may be a relatively biocompatible polymer, such as polycaprolactone. A photoresist may be any photoresist capable of being patterned photolithographically to yield a porous scaffold on which TM, MVE, or SC cell-like cells can grow. A photoresist may be, for example, an SU-8 photoresist, KMPR®, or ORMOCORE™.

Pores in a porous scaffold may provide a three-dimensional structure for cells to attach to and grow on to permit more physiological morphology, structure, and biochemical profile expression compared to growth on a two-dimensional surface. A porous scaffold may have a plurality of pores therethrough. A micropatterned porous substrate is a substrate in which patterns on a micron-scale, such as openings in the substrate surface that are between 1 μm and 50 μm in width, or sub-micron-scale, such as openings in the substrate surface that are hundreds of nm in width, have been made. For example, a pattern of pores with dimensions within such range may be formed. Pores may be any cross-sectional shape desirable, such as circular, square, triangular, rectangular, or irregularly shaped. They may have any average width or diameter desirable. In some examples, they may have widths on the order of 1 μm to 20 μm. For example, pore widths may be between approximately 5 μm and 15 μm. They may also be between 5 μm and 10 μm, between 10 μm and 15 μm, or between 15 μm and 20 μm. In one example, pore widths may be between approximately 11 μm and 13 μm. Where pores do not all have exactly the same width as each other, they may nevertheless have a width that is within 1 μm or less of an average pore width.

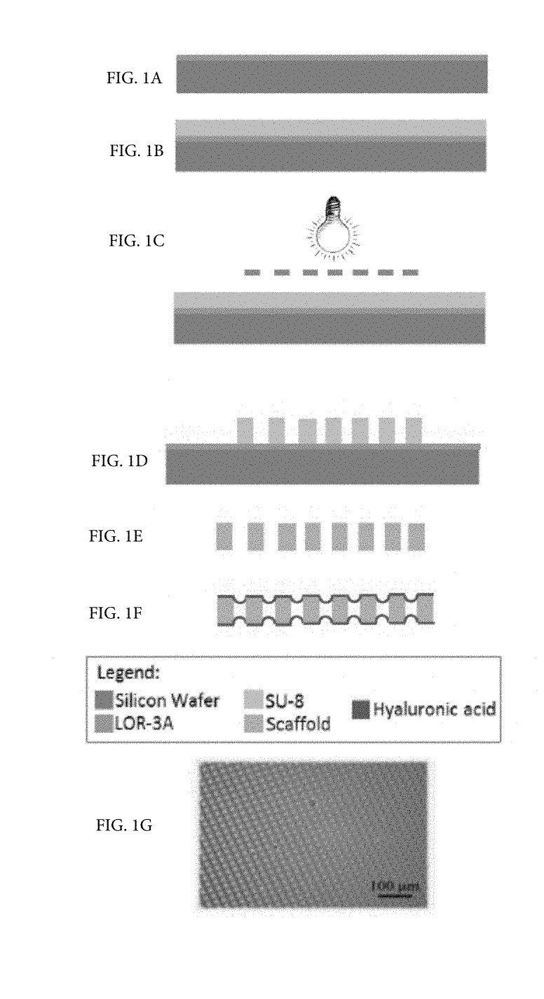

Density of pores in a porous scaffold may be determined by specifying a space between pores, referred to as beam width. When pores are regularly spaced throughout a porous scaffold, a beam width may be relatively constant throughout a porous scaffold. In other examples, where pores are distributed in a less orderly manner, beam width may differ throughout a porous scaffold. A beam width may be anywhere from less than 1 μm to more than 15 μm. A beam width may be between 1 μm and 5 μm, between 5 μm and 10 μm, or between 10 μm and 15 μm. In an example, a beam width may be between 7 μm and 8 μm. Where adjacent pores do not all have exactly the same beam width between them, they may nevertheless have a beam width that is within 1 μm or less of an average beam width. Together beam width and pore width may determine the most desirable patterning on a surface of a porous scaffold for the growth and, if preferred, confluence of cells grown on a surface thereof. Any one of these beam widths may be combined with any one of the pore widths disclosed above, depending on the desired pore volume fraction and pore density. An example of a porous scaffold with a regular array of pores with substantially consistent pore widths and beam widths is shown in FIG. 1G.

A porous scaffold may be any thickness desired. In some examples, a thickness may be chosen that permits cells growing at opposite ends of pores (i.e., on opposing surfaces of the substrate) to extend, protrude, or migrate towards each other and contact each other. As referred to herein, contact may be direct, as in cell membrane-to-cell membrane contact. Or it may be indirect, such as if there is some biological or other component, such as extracellular matrix or components of extracellular matrix, between two cells that is contacted by them both. A scaffold may be less than 200 nm, between 200 nm to 1 μm, or between 1 μm and 40 μm thick or more. In some examples, a porous scaffold may be approximately 5 μm, 10 μm, 15 μm, 20 μm, 25 μm, 30 μm, or 35 μm thick. An example of porous scaffold thickness is shown in FIG. 1F, which is a SEM image of a cross section of a porous scaffold. Any one of these porous scaffold thicknesses may be combined with any one of the pore volume fractions or pore densities created by modifying pore width and beam width, in accordance with the pore widths and beam widths given above. Every possible permutation of combinations and subcombinations of these features is hereby explicitly included within the present disclosure.

A porous scaffold and pores therein may be formed by any suitable method for obtaining a desired porous scaffold thickness, pore width, and beam width. A porous scaffold may be cast, or molded. It may be manufactured from a solid structure that is surface-etched to create pores. In an example, a porous scaffold is fabricated from a photoresist. For example, a negative photoresist layer may be deposited on a surface (FIGS. 1A and 1B), and a photomask patterned above it (FIG. 1C). In another embodiment, a positive photoresist could be used instead of or, if multiple photolithographic steps were used to pattern a porous scaffold, a negative photoresist. When regions of the negative photoresist are exposed to electromagnetic radiation such as ultraviolet light, regions not blocked by the photomask become cross-linked upon exposure to the electromagnetic radiation (FIG. 1C). Subsequent baking and developing of the photoresist results in formation of a scaffold, and portions that had been covered by the photomask and were therefore not cross-linked are washed away, leaving pores through a scaffold (FIG. 1D). In an example, an SU-8 photoresist is used for fabrication of a porous scaffold, but any other suitable photoresist may be used in the same manner optionally, removal of a porous scaffold formed from a negative photoresist from the surface on which the photoresist layer was deposited before photolithographic processing may be facilitated by depositing a release layer between the photoresist layer and the surface (e.g., OMNICOAT™) (FIG. 1A). In some examples, a plasticizer may be used to modify the strength, pliability, or other mechanical properties of the porous scaffold.

The surface of a porous scaffold may be modified or coated to improve cell adhesion to and growth on a porous scaffold's surface. A surface of the porous scaffold may be covalently modified by the covalent addition of a chemical moiety that promotes cell viability, growth, or adhesion. A surface of a porous scaffold may also be coated with a layer that promotes cell adhesion, viability, or growth. A coating to be or contain poly-L-lysine, gelatin, a hyaluronic acid-based polymer, a hydrogel, a hyaluronic acid-based hydrogel (e.g., EXTRACEL™), collagen, fibronectin, vitronectin, chitosan, extracellular matrix, an RGD-containing peptide, peptide attached to acrylate (e.g., SYNTHEMAX®), or combinations of any of these. Any surface modification, or surface coating, may be combined with any of the pore widths, beam widths, and porous scaffold thicknesses described above. And any of these may be combined with any thermoset, thermoplastic, or photoresist porous scaffold, depending on the desired characteristics and intended use. Every possible permutation of combinations and subcombinations of these features is hereby explicitly included within the present disclosure.

A porous scaffold may be cut or formed into desired shapes and sizes. It may be maintained as a substantially flat scaffold or in configured into curved, folded, angled, or other three-dimensional shapes. It may have been manufactured in such shapes or, if pliable after manufacture, physically manipulated into such shape. It may also be cut or sized as appropriate for use in standard or specially designed laboratory materials such as multi-well culture plates. A circle of porous scaffold may be formed and sized so as to fit within a well of a standard 6-well plate, or a well of any other preferred size. To facilitate handling of porous scaffold without requiring direct contact with cells that may be growing on it, a porous scaffold may be adhered to, attached to, or contained in a holder, such as a collar or rim around the edge of the scaffold. For example, aluminum tape or other material may be placed around the rim of a porous scaffold to form a collar. Manipulation of the placement, orientation, and positioning of a scaffold contained in such a collar may then be accomplished by grasping and moving the collar and the porous scaffold therein. For suspension of a scaffold above a surface on which it is disposed, a collar may extend some distance above and below the surfaces of the porous scaffold such that placement of an open face of the collar on a surface results in suspension of the porous scaffold above such surface. The pore width ranges recited above, beam width ranges recited above, scaffold thicknesses recited above, and surface modifications and coating recited above, may all be used in combination with the shaping and collaring of porous scaffolds as disclosed here. Any appropriate pore width may be combined with any appropriate beam width, which combination may be combined with any appropriate scaffold width, which combination may, optionally, be combined with any surface treatment or coating, which may optionally be combined with shaping and/or configuring a porous scaffold. Every possible permutation of combinations and subcombinations of these features is hereby explicitly included within the present disclosure.

TM cells, SC cells, or MVE cells may be grown on each surface of a porous scaffold. Seeding cells on one surface, followed by incubation in physiologically appropriate conditions and medium, may permit growth of cells on the surface. Cells may also protrude into pores. The three-dimensional arrangement of cells on a porous scaffold permits more physiological phenotypes of these cells that growth on a flat, two-dimensional surface, permitting a more reliable model of conventional outflow tract function. Cells may be seeded and grown on one side of a scaffold and allowed to grow for one period of time. Subsequently, cells may be seeded on the other surface, permitting co-culture of cells, one population on one surface of the scaffold and another population on the other surface of the scaffold, opposite the first. Two different types of cells may be seeded and grown on each side, the same cell type may be seeded and grown on both sides, or mixtures of the same or different cells types, in the same or different ratios, may be seeded and grown on each side.

For example, cells in a density of 10,000 cells/cm² or higher, 20,000 cells/cm² or higher, 30,000 cells/cm² or higher, or 40,000 cells/cm² or higher may be seeded and grown one side of a porous scaffold and allowed to grow for a period of time which the porous scaffold is positioned with the surface facing away from a substrate on which the porous scaffold is set, optionally suspended by a collar. After the period of time, which may be on the order of a day, days, or a week or more, the porous scaffold may be inverted, or flipped over, so that the cells grown on the first surface are now facing the substrate on which the scaffold is disposed. A second population of cells may now be seeded on the second surface and allowed to grow for a period of time. For example, TM cells may be seeded on one surface and allowed to grow for a period of time, such as seven days, after which the scaffold is flipped and MVE cells are then seeded on the second surface and allowed to grow for a period of time. Seeded cells attach to the porous scaffold at their basal surfaces.

A result of seeding populations of cells on opposing surfaces of a porous scaffold in accordance with aspects of the present disclosure is that they establish polarity relative to their orientation from the porous scaffold surface on which they are grown. Cells will adhere to the porous scaffold with their basal membranes. When cells are seeded on opposite surfaces, their basal membranes face each other through the porous scaffold, recapitulating the physiology of the conventional outflow tract. When TM cells are seeded and grown on one surface of a porous scaffold and MVE cells are co-cultured with the TM cells on the other surface of the porous scaffold and induced to become SC cell-like cells, the basal membranes of each cell type will be oriented towards each other's.

Cells may be seeded at a density of 40,000 cells/cm², or any of the other density preferred. Any such density could be seeded on a porous scaffold with a regular or irregular array of pores, with: pore widths of between approximately 5 μm and 15 μm, between 5 μm and 10 μm, between 10 μm and 15 μm, between 15 μm or 20 μm, or between approximately 11 μm and 13 μm; beam widths of between 1 μm and 5 μm, between 5 μm and 10 μm, between 10 μm and 15 μm, or between 7 μm and 8 μm; scaffold thickness of between 1 μm to 40 μm thick or thicker, 5 μm, 10 μm, 15 μm, 20 μm, 25 μm, 30 μm, or 35 μm thick; none or one or more of any of the surface modifications or coatings identified above (poly-L-lysine, gelatin, a hyaluronic acid-based polymer, a hydrogel, a hyaluronic acid-based hydrogel (e.g., EXTRACEL™), collagen, fibronectin, vitronectin, chitosan, extracellular matrix, an RGD-containing peptide, a peptide attached to acrylate (e.g., SYNTHEMAX®)). Any combination of these features may be combined with a scaffold of any disclosed material, size, or shape, collared or not. And disclosed cell seeding density or range thereof and periods of growth permitted between seedings may be used in combination with any of the foregoing combinations. Every possible permutation of combinations and subcombinations of these features is hereby explicitly included within the present disclosure.

In accordance with the present disclosure, co-culturing TM cells on one surface of a porous scaffold and MVE cells on another surface of a porous membrane induces MVE cells to adopt a phenotype similar to that of SC cells. MVE cells co-cultured with TM cells in this manner may adopt a cellular morphology, cytoskeletal structure, and/or patterns of expression of biomarkers, that is characteristic of SC cells. MVE cells that have undergone such a transformation are referred to herein as SC cell-like cells. As a non-limiting example, SC cell-like calls may also refer to Schlemm's canal cells, which are SC cell-like cells by nature of exhibiting morphological, biochemical, and/or other indicia typical of Schlemm's canal cells. SC cells can be difficult to obtain reliably and in desirably high volume. MVE cells and TM cells, by comparison, may be relatively easily obtained. By creating SC cell-like cells by co-culturing MVE cells with TM cells and using them as a proxy for SC cells, a system for modeling conventional outflow track function may be made and used, such as for studying pathology of IOP-related disorders and developing preventions and treatments therefore.

A porous scaffold on which co-cultured TM cells and SC cell-like cells have been induced to develop in accordance with aspects of the present disclosure may be evaluated for morphology, cytoskeletal arrangement, biomarker expression, as well as various physiological functions, for modeling a conventional outflow tract. SC cells may express biomarkers such as prospero homeobox protein 1 (PROX1), Platelet/Endothelial Cell Adhesion Molecule 1 (PECAM1), VE-cadherin, fibulin-2, vascular endothelial growth factor receptor 2 (VEGFR-2), ephrin B2 (EFNB2), CD34, TIE2, or Fms-Related Tyrosine Kinase 4 (FLT4), in levels or combinations that are not conventionally expressed by MVE cells. In culture, such as on a porous scaffold, SC cells may exhibit polygonal, cobblestone-like morphology and form a thin monolayer with distinct junctions formed between cells, while TM and conventional MVE cells may be elongated and have a rougher cell topography. When MVE cells are co-cultured with TM cells on porous scaffolds in accordance with aspects of the present disclosure, they may adopt a morphology similar to that exhibited by SC cells, observable under microscopy, of polygonal, cobblestone-like morphology and form a thin monolayer with distinct junctions formed between cells, a characteristic of SC cell-like cells. They may also exhibit expression patterns of biomarkers that differentiates them from traditional MVE cells and resemble SC cells.

Gene or protein expression of such markers may be measured immunohistochemically, by in situ hybridization histochemistry, quantitative PCR, western blotting, through the use of fluorescently tagging the gene product recombinantly or otherwise, or any other well-known method suitable for distinguishing and comparing expression patterns between MVE, SC, TM, and SC cell-like cells. When MVE cells have been co-cultured with TM cells on opposing surfaces of a porous scaffold in accordance with aspects of the present disclosure for a duration of time sufficient for them to exhibit a morphology characteristic of SC cells, or a cytoskeletal structure characteristic of SC cells, or expression of a biomarker or pattern of biomarkers characteristic of SC cells that distinguished MVE cells grown on a porous scaffold on their own from those co-cultured on a porous scaffold with TM cells in accordance with aspects of the present disclosure, they are CS cell-like cells. Measurement of any of the morphological or molecular indicia of adoption of an SC cell-like nature identified here, or others known to skilled artisans to evince an SC cell typology as distinguished from a MVE cell typology may be used in accordance with the present disclosure in like manner as use of such indicia is described above. Any combination of these indicia may be used, together with any of the pore sizes, porous scaffold widths, beam widths, scaffold materials, scaffold surface modifications or coatings, cell seeding densities, or sizes, shapes, or configurations of porous scaffolds as may be deemed preferable in a given circumstance and be within the range of examples according to the present disclosure. Every possible permutation of combinations and subcombinations of these features is hereby explicitly included within the present disclosure.

TM cells and SC cell-like cells co-cultured on a porous scaffold in accordance with aspects of the present disclosure may serve as a system to model conventional outflow tract function, physiology, and pathophysiology. FIG. 13A is a diagrammatic representation of a testing apparatus for performing perfusion-related testing in accordance with aspects of the present disclosure. A syringe pump or other pressure source may feed into a holder for a porous scaffold, with TM and SC cell-like cells co-cultured thereupon, with pressure controlled and measured by a pressure transducer. The pressure across the porous scaffold and cells, and other aspects related to pressure such as through-flow, flow rate, resistance to flow, hydraulic conductivity, and other indicia of conventional outflow tract function may be measured. For example, fluids can be perfused across the co-culture-bearing scaffold and aspects of fluid mechanics measured as an indication of conventional outflow tract function. In an example, perfusion flow may first go in the apical-to-basal direction across the surface of the porous scaffold bearing TM cells, through the porous scaffold, then through the surface of the porous scaffold bearing SC cell-like cells, in the basal-to-apical direction. Aspects of membrane pressure, outflow dynamics, and cellular function can similarly be measured under different conditions known or hypothesized to replicate disease states, or lack thereof, or conditions known or believed to prevent, ameliorate, or cure disease or pathological states. In an example, fluid may be perfused across a porous scaffold bearing co-cultured TM and SC cell-like cells and effects of transmembrane pressure, flow rate, through-flow, resistance to flow, hydraulic conductivity, vacuole dynamics, pore formation, or outflow facility of the co-cultured TM cells and SC cell-like cells may be measured. Mechanisms of vacuole formation and function and of pore formation in aspects of conventional outflow tract may be related to the pathophysiology of IOP and related disease states. Using TM cell and SC cell-like cells co-cultured on a porous scaffold in accordance with aspects of the present disclosure, vacuole dynamics and function and pore formation and underlying mechanisms may be studied, such as in response to perfusion. Other aspects of cellular physiology or function may similarly be measured, in response or relation to perfusion or otherwise. Electrical conductivity, membrane potential, and other aspects of cell layer, cell membrane, or cell physiology, such as transmembrane pressure, transcellular pressure, and trans-tissue pressure, may be used as appropriate to the purposes for which a system as disclosed herein is used.

Also in accordance with aspects of the present disclosure, SC cell-like cells, and a model of conventional outflow tract of which they are a part, may be treated pharmacologically or transfected so as to manipulate gene or protein expression and the effects of such manipulations on the functional attributes enumerated above, or expression patterns of genes or proteins of interest, identified in response to such treatment. For example, a known risk factor for development of IOP-related disease may be applied and the functional, physiological, or molecular consequences of such manipulation measured to provide indications of the pathophysiological processes that may underlie the disease state of interest, such as, for example, glaucoma. Similarly, treatments known or believed to prevent or treat such diseases may be applied and tested for whether they enhance or improve functional read-outs of the porous scaffold bearing TM and SC cell-like cells in co-culture, or prevent effects of known or suspected risk factors for disease. In this manner, in one aspect, the system for modeling conventional outflow tract function of the present disclosure may serve as both an investigative tool for understanding the physiology of normal outflow tract tissue or pathophysiology of IOP-related diseases such as glaucoma and also as a mechanism for screening potential pharmacological or other treatments or preventative measures for such disorders, or testing for potentially harmful effects of environmental or other compounds such as toxicants and food additives, or for unwanted side-effects of medications used for treating or preventing other pathophysiological states.

Skilled artisans would appreciate that different combinations of cell types disclosed herein could be used in, on, or with the systems, methods, or apparatuses disclosed herein and fall within aspects of the present disclosure. For example, a scaffold could have trabecular meshwork cells in combination with microvascular endothelial cells, Schlemm's canal cell-like cells, Schlemm's canal cells, or any combination of the latter three in accordance with the present disclosure. In some aspects, a scaffold may contain only one such cell type, whereas in others in may have 2 or more. Similarly, stem cells that give rise to trabecular meshwork cells, microvascular epithelial cells, Schlemm's canal cell-like cells, or Schlemm's canal cells may be used, as may precursor cells to each of the foregoing. One or any combinations of two or more of the foregoing may be used, in keeping with the present disclosure. All permutations and combinations of such cell type usages, together with the variations in scaffold, coating, and cell treatment and measurement, are explicitly included with the present disclosure.

### EXAMPLES

Aspects of the present disclosure now will be further illustrated by, but by no means are limited to, the following Examples.

Primary Human Trabecular Meshwork Cell Culture

Human TM (HTM) cells were isolated from donor tissue rings discarded after penetrating keratoplasty. Isolation and culture conditions were as previously described and available in published literature, such as in Polansky, J. R., et al., Human trabecular cells. I. Establishment in tissue culture and growth characteristics. Investigative ophthalmology & visual science, 1979. 18(10): p. 1043-9; and Steely, H. T., et al., The effects of dexamethasone on fibronectin expression in cultured human trabecular meshwork cells. Investigative Ophthalmology & Visual Science, 1992. 33(7): p. 2242-50. Before use in experiments, all HTM cell strains were characterized by expression of αB-crystalline and α-smooth muscle actin. HTM cells were initially plated in 75 cm² cell culture flasks with 10% fetal bovine serum (FBS) (Atlas Biologicals, Fort Collins, Colo.) in Improved MEM (IMEM) (Corning Cellgro, Manassas, Va.) with 1% 10 mg/mL gentamicin. Fresh medium was supplied every 48 hrs. Cells were maintained at 37° C. in a humidified atmosphere with 5% carbon dioxide until 80-90% confluence which point cells were trypsinized using 0.25% trypsin/0.5 mM EDTA (Gibco, Grand Island, N.Y.) and subcultured. At least three donors' human primary cell cultures were used during experiments. All studies were conducted using cells before the 5th passage.

Primary Human Microvascular Endothelial Cell Culture

Primary human microvascular endothelial cells isolated from neonatal dermis, were purchased from LifeTechnologies (Carlsbad, Calif.). The HTM cells were initially plated in Attachment Factor (LifeTechnologies, Carlsbad, Calif.)-coated 25 cm² cell culture flasks and cultured in 131 Medium supplemented with Microvascular Growth Supplement (MVGS) (Life Technologies, Carlsbad, Calif.). Fresh culture medium was supplied every 48 hrs. Cells were maintained at 37° C. in a humidified atmosphere with 5% carbon dioxide until 80%-90% confluence at which point cells were trypsinized using 0.25% Trypsin/0.5 mM EDTA and subcultured. All studies were conducted using cells before the 4th passage.

Primary Human Schlemm's Canal Cell Culture

Primary human Schlemm's canal cells were isolated as described in publicly available literature. Stamer, W. D., et al., Isolation, Culture, and Characterization of Endothelial Cells from Schlemm's Canal. Investigative Ophthalmology & Visual Science, 1998. 39(10): p. 1804-12. HSC cells were initially plated in 75 cm² cell culture flasks with 10% Premium Select Fetal bovine serum (PFBS; Atlanta Biologicals, Lawrenceville, Ga.) in Dulbecco's modified Eagle's medium (DMEM; Life Technologies, Carlsbad, Calif.) supplemented with penicillin (100 units/mL), streptomycin (0.1 mg/mL), and L-glutamine (0.292 mg/mL; Life Technologies, Carlsbad, Calif.). Fresh medium was supplied every 48 hrs. Cells were maintained at 37° C. in a humidified atmosphere with 5% carbon dioxide until 80-90% confluence which point cells were trypsinized using 0.25% trypsin/0.5 mM EDTA (Gibco, Grand Island, N.Y.) and subcultured. All studies were conducted using cells before the 4th passage.

Biomimetic Scaffold Fabrication

SU-8 2010 (MicroChem Corp.) was used to develop free-standing biomimetic porous microstructures that served as scaffolds on which primary HTM cells were cultured. Scaffolds were fabricated using standard photolithographic techniques as previously described in publicly available literature and in accordance with the procedure illustrated in FIG. 1. Torrejon, K. Y., et al., Recreating A Human Trabecular Meshwork Outflow System On Microfabricated Porous Structures. Biotechnology and Bioengineering, 2013. 110(12): p. 3205-18. Briefly, a release layer was spin-coated on the wafer and baked at 150° C. SU-8 2010 was applied by spin-coating to final thickness of 5 μm, then baked at 95° C. and cooled to room temperature. The resist was UV-exposed through a mask containing the desired pattern, baked at 95° C. and developed in PGMEA developer (MicroChem Corp.). SU-8 scaffolds with the desired features were released from the substrate, washed with acetone and sterilized using 70% ethanol. In some examples, Sterile SU-8 scaffolds with 12 μm pores were coated with HA-based hydrogel coating. Briefly, HYSTEM-C HYDROGEL KIT® (ESI BIO, Alameda, Calif.) which contained thiol-modified sodium hyaluronate (GLYCOSIL®), thiol-modified gelatin (GELIN S®) and polyethelyne glycol diacrylate (EXTRALINK®), were prepared in solution as per manufacturer's instructions. To form the hydrogel, GLYCOSIL® and GELIN S® (1:1) were mixed, followed by addition of EXTRALINK® at 1:4 volume ratios (EXTRALINK®: GELIN S®+ GLYCOSIL®). Porous SU-8 scaffolds were then dipped in the final hydrogel solution; excess solution was removed from the scaffolds and allowed to gel at room temperature for 10-20 minutes. In other examples, porous SU-8 scaffolds were also coated with gelatin, which was also effective.

Culture of HSC and HMVEC Cells on Biomimetic Scaffolds

40,000 human MVE cells (HMVEC) or human SC (HSC) cells were seeded on HA-coated SU-8 scaffolds and cultured for 7 or 14 days. HMVEC and HSC cells were cultured in 131 Medium supplemented with MVGS (LifeTechnologies, Carlsbad, Calif.) or DMEM supplemented with 10% PFBS, respectively. Additional media were used during experiments aimed towards studying the effect of biochemical stimuli on HMVEC cultures, including; 10% bovine aqueous humor in DMEM and HTM cell conditioned medium.

3D Culture of HTM/HMVEC and HTM/HSC on Scaffolds

To create 3D constructs, 40,000 HTM cells were seeded on each microfabricated SU-8 scaffold in a well of a 24-well plate and cultured in 10% FBS-DMEM for 7 days. Medium was changed every 2-3 days. HTM constructs were then inverted and HMVEC or HSC cells (40,000 cells/sample) were seeded and cultured for additional 7 to 14 days.

Scanning Electron Microscopy

HTM, HMVEC, HSC, HTM/HSC and HTM/HMVEC samples were fixed with 3% glutaraldehyde solution in 0.1 M phosphate buffer containing 0.1 M sucrose for 2 hrs at room temperature. The samples were then rinsed three times in 0.1 M phosphate buffer, dehydrated in a graded ethanol series (50%, 70%, 80%, 95% and 100% ethanol) and slowly infiltrated with a graded hexamethyldisilazane (HMDS)-ethanol series (25%, 50%, 75%, 100% HMDS for 5, 5, 10, and 10 minutes; respectively) for drying, and then samples were sputter-coated to prevent charging. Samples were observed under a LEO 1550 field emission scanning electron microscope (SEM) (Leo Electron Microscopy Ltd, Cambridge, UK) as described previously [22]. All images were taken using the same electron beam energy.

Immunocytochemistry Followed by Confocal Microscopy

After 7 or 14 days in culture, constructs were fixed, permeablized, and stained for F-actin cytoskeleton using phalloidin (Life Technologies) or incubated with antibodies against HTM markers myocilin, and αB-crystallin, HSC-marker PECAM-1, VE-cadherin and fibulin-2, and ECM proteins, collagen type IV, fibronectin and laminin (Abcam) as described in publicly available literature. Torrejon, K. Y., et al., Recreating a Human Trabecular Meshwork Outflow System On Microfabricated Porous Structures. Biotechnology and Bioengineering, 2013. 110(12): p. 3205-18. Primary antibodies used are shown in Table 1.

TABLE 1
Primary antibodies used during this study.

| | | | | Secondary |
|---|---|---|---|---|
| Name | Specie | Dilution | Company | Antibody |
| Alexa-488 | — | 1/100 | Life | — |
| conjugated | | | Technologies | |
| Phelioidin | | | | |
| CD-31 | mouse | 1/200 | Abcam | Goat anti-mouse 594 |
| (PECAM-1) | | | | |
| Fibulin-2 | Goat | 1/200 | Santa Cruz | Donkey anti-goat 647 |
| | | | Biotechnology | |
| VE-cadherin | Rabbit | 1/500 | Cell Signaling | Goat anti-rabbit 488 |
| Collagen | Rabbit | 1/500 | Abcam | Goat anti-rabbit 488 |
| type IV | | | | |
| Fibronectin | Mouse | 1/200 | Abcam | Goat anti-mouse 594 |
| Laminin | Chicken | 1/500 | Abcam | Goat anti-chicken 633 |

Appropriate secondary antibodies were used for each protein (Table 1). Laser scanning confocal microscopy was performed using a Leica SP5 confocal microscope, and images were acquired at 40× and 63× magnifications with an oil-immersion objective. Confocal images were processed using Leica LasAF software, and all confocal images within a given experiment were captured using the same laser intensity and gain settings in order to be able to compare intensities across samples.

Vacuole Induction

A perfusion apparatus was used as described in publicly available literature and in accordance with the apparatus illustrated in FIG. 13. A. Torrejon, K. Y., et al., Recreating a Human Trabecular Meshwork Outflow System On Microfabricated Porous Structures. Biotechnology and Bioengineering, 2013. 110(12): p. 3205-18. HTM/HSC and HTM/HMVEC samples were securely placed in the perfusion chamber and perfused at 5 μl/min for 2 hrs. Samples were perfused in an apical-to-basal direction across the surface of the porous scaffold bearing TM cells, through the porous scaffold, then in a basal-to-apical direction through the surface of the porous scaffold bearing SC cell-like cells, with perfusion medium consisting of Dulbecco's modified Eagle's medium (DMEM) (Cellgro) with 0.1% gentamicin (MP). The temperature was maintained at 34° C. The perfusate was then switched to 3% glutaraldehyde solution in 0.1 M phosphate buffer containing 0.1 M sucrose (SEM fixative). Fixed samples were then dehydrated and prepared for SEM imaging. Vacuole/pore size were measured using the embedded LEO electron microscopy image software. Four different samples per condition including HSC cells from two donors and HTM from three donors were studied for these experiments. HTM/HMVEC showed similar trans-tissue pressure to 3D HTM/HSC constructs and higher than 3D HTM alone constructs at constant flow 5 μl/min suggesting that HMVECs can simulate the HSC flow resistance in the 3D artificial system. FIG. 13B.

Quantitative Real-Time PCR (qPCR) Analysis

Total RNA was extracted from samples using an RNeasy Plus Mini kit (Qiagen Inc., Valencia, Calif.). RNA concentrations were determined using a NanoDrop spectrophotometer. 50 ng of RNA per sample was used for each qPCR experiments. qPCR was carried out using TAQMAN RNA-TO-CT™ 1-Step Kit (Applied Biosystems, Carlsbad, Calif.) and performed on an AB StepOnePlus Real Time PCR system (Life Technologies, Carlsbad, Calif.) using primers for αB-crystallin, PECAM-1, fibulin-2, collagen type IV, fibronectin, laminin and GAPDH. Primer sequences are provided in Table 2.

TABLE 2
Primer sequences used in qPCR analysis.

| NCBI | | | |
|---|---|---|---|
| Gene Symbol | Ref. Seq. number | Forward Primer | Reverse Primer |
| CRYAB | NM_001885 | SEQ ID NO: 1 | SEQ ID NO: 2 |
| PECAM-1 | NM_000442 | SEQ ID NO: 3 | SEQ ID NO: 4 |
| FBLN2 | NM_001165035 | SEQ ID NO: 5 | SEQ ID NO: 6 |
| COL4A1 | NM_001845 | SEQ ID NO: 7 | SEQ ID NO: 8 |
| FN1 | NM_054034 | SEQ ID NO: 9 | SEQ ID NO: 10 |
| LAMA4 | NM_001105206 | SEQ ID NO: 11 | SEQ ID NO: 12 |
| GAPDH | NM_002045 | SEQ ID NO: 13 | SEQ ID NO: 14 |

Primer nucleotide sequences were as follows:

SEQ ID NO: 1
CAATCACATCTCCCAACACCT;
SEQ ID NO: 2
CTGGTTTGACACTGGACTCTC;
SEQ ID NO: 3
ATTGCTCTGGTCACTTCTCC;
SEQ ID NO: 4
CAGGCCCATTGTTCCC;
SEQ ID NO: 5
CCAGGCACTCGTCATTGTC;
SEQ ID NO: 6
CCAACTCTGTCCATTCTATCCC;
SEQ ID NO: 7
CCTTTGTGCCATTGCATCC;
SEQ ID NO: 8
GAACAAAAGGGACAAGAGGAC;
SEQ ID NO: 9
GTCCTTGTGTCCTGATCGTTG;
SEQ ID NO: 10
AGGCTGGATGATGGTAGATTG;
SEQ ID NO: 11
AGTGCTCTCCTGTTGTGTTC;
SEQ ID NO: 12
GAATGTGTGCCCTGCGA;
SEQ ID NO: 13
TGTAGTTGAGGTCAATGAAGGG;
SEQ ID NO: 14
ACATCGCTCAGACACCATG.

The temperature profile was as follows: 48° C. for 15 min (reverse transcription step), followed by an enzyme activation step of 95° C. for 10 min, 40 cycles of 15 s denaturation at 95° C. and 1 min anneal/extend at 60° C. Relative quantitation data analysis was performed using the comparative quantification method, ΔΔCt, with GAPDH as the endogenous reference. All samples were normalized to GAPDH. q-PCR experiments were performed in triplicate (technical replicates) and from duplicate biological experiments using three donor cells. Average values are presented as mean±SD.

Statistical Analysis

Data were expressed as the average±standard deviation. The difference between HTM/HSC and HTM/HMVEC samples was analyzed using two-way ANOVA followed by Bonferroni post-tests (GraphPad Prism 6.02; GraphPad Software, Inc., La Jolla, Calif.). P values P<0.05 and P<0.001 considered significant and highly significant, respectively.

Cultivation and Characterization of HMVECs Grown on Micropatterned, Well-Defined, Porous SU-8 Scaffolds

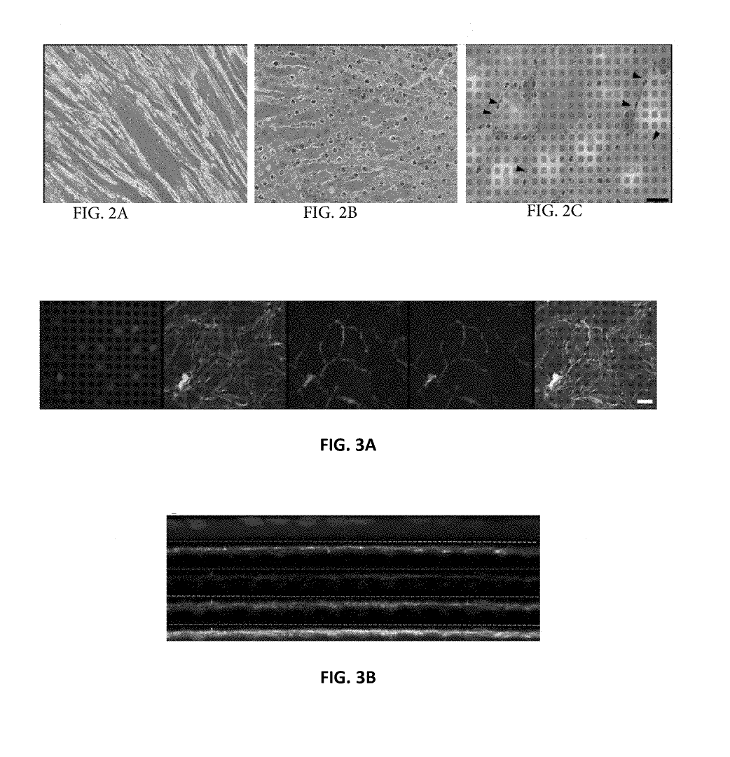

Compared to HTM and HSC cells, HMVECs present polygonal, cobblestone-like morphology and form a thin cell monolayer with distinct junctions formed between cells, while HTM and HSC cells were elongated and have rougher cell topography. FIG. 2A, FIG. 2B, and FIG. 2C show scanning electron micrographs of HTM, HSC, and HMVEC cells, respectively, cultured on scaffolds for 14 days. Furthermore, a HTM cell layer was distinctively thicker than a HMVEC and HSC layer, appearing less translucent under the same electron beam energy in the SEM.

HMVEC's polygonal morphology was confirmed by phalloidin-stained F-actin expression, revealing the stress actin fibers that make up the cytoskeleton. Many of these actin fibers appeared crosslinked, forming vertices and bundles and interconnecting multiple F-actin fibers at one point. FIG. 3A shows top-down Z-stack confocal imaging of HMVEC's grown on SU-8 scaffolds (from left to right, DAPI, F-actin cytoskeleton, PECAM-1, fibulin-2). Analysis of HMVEC protein expression demonstrated that these cells maintained their vascular markers and expressed adherence proteins such as PECAM-1. FIG. 3. PECAM-1 was expressed throughout these cells and mostly also in the intercellular spaces or junctions between cells. Fibulin-2, a secreted glycoprotein, was also expressed by HMVECs and its pattern appeared similar to that of PECAM-1. Cross-sectional confocal imaging of HMVEC cells grown on SU-8 scaffolds, shown in FIG. 3B (top to bottom: DAPI, F-actin cytoskeleton, PECAM-1, and fibulin-2), revealed fibulin-2 diffused throughout the thickness of the cell layer and mostly basal and extracellular expression compared to PECAM-1. These results demonstrated the feasibility of culturing HMVECs on HA-coated SU-8 scaffolds, maintaining their innate cobblestone morphology and endothelial markers. The morphology of these cells cultured alone differed greatly from HTM and HSC cells. FIGS. 2A-C.

Differential Expression of HSC-Marker Proteins in HMVECs Cultured on SU-8 Scaffolds

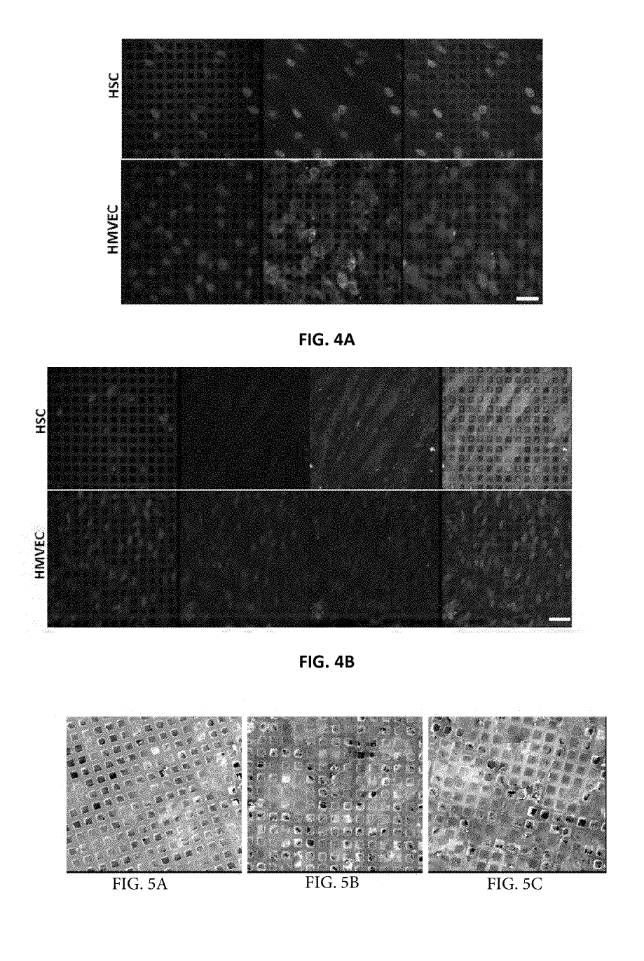

HMVEC and HSC cell-marker expression were compared to investigate their similarities and differences. To characterize the common markers between these two cell types, immunocytochemistry against HSC-markers after 7 days of cell culture was used, including VE-cadherin, PECAM-1 and fibulin-2, as shown in FIGS. 4A-B. HSC cells are shown in the top panels, and HMVEC cells are shown in the bottom panels, of FIGS. 4A and 4B, The left, middle, and right panels in FIG. 4A show DAPI, VE-cadherin, and merged immunocytochemistry, respectively. Panels in FIG. 4B, from left to right, show DAPI, PECAM-1, fibulin-2, and merged. Additionally, as a negative control and to distinguish potential contamination during isolation of HSCs, cells were stained for the HTM specific marker αB-crystallin. HSC cells expressed VE-cadherin greatly in the nuclei as well as at the edges of the cells, with a spindle-like appearance mostly found towards the thin edges of the cells. FIG. 4A, top panel. HMVECs expressed VE-cadherin in a different pattern, mostly towards the edges of these round cells. FIG. 4A, bottom panel. In particular, no nuclear expression of VE-cadherin was observed in HMVECs. The opposite was revealed when comparing PECAM-1 expression in HMVECs and HSC cells. Mostly nuclear expression of this protein was observed in HMVECs while HSCs expressed cytoplasmic diffused PECAM-1, delineating the elongated morphology of these cells. FIG. 4B. Fibulin-2 was also expressed in both cell types. FIG. 4B. In HSCs, fibulin-2 was expressed extracellularly all through the scaffolds. HMVECs expressed fibulin-2 to a lesser extent than that seen in HSCs, and it appeared to be mostly secreted under the nuclear areas of these cells. αB-crystallin, used as negative control since this protein is expressed in HTM, was not expressed by neither HSCs nor HMVECs. The results presented in this section demonstrated that despite morphological differences, HMVECs express HSC-markers. There are differences in the expression pattern of VE-cadherin, PECAM-1 and fibulin-2.

Feasibility of Differentiating HMVECs into HSC-Like Cells Using Exogenous Biochemical Cues

The effect of exogenous biochemical cues on HMVEC growth and potential HSC-like morphogenesis, including HMVEC medium, HTM medium, HTM cell-conditioned medium, and aqueous humor, was tested. HMVEC medium was used as a negative control, which is the standard medium for endothelial cell maintenance and is composed of medium 131, supplemented with MVGS (4.9% FBS, 1 μg/mL hydrocortisone, 3 ng/mL human fibroblast growth factor, 10 μg/mL heparin, 1 ng/ml human epidermal growth factor and 0.08 mM dibutyryl cyclic AMP). HTM medium, which is used to maintain HTM morphology and function and composed of 10% FBS in DMEM, was used to see if it is sufficient to induce HSC-like morphology. Given that HTM cells are known to secrete several cytokines and growth factors into their microenvironment, cell-conditioned medium was also used to provide biochemical cues for potential HMVEC differentiation. FIGS. 5A-5C show scanning electron micrographs of the effects of media on HMVEC differentiation. After 7 days of culture, SEM analysis demonstrated HMVECs reached confluence and retained their innate cobblestone morphology in HMVEC medium (FIG. 5A), HTM medium (FIG. 5B) and HTM cell-conditioned medium (FIG. 5C). HMVECs grown in HTM cell-conditioned medium were also noted to appear slightly thicker than those grown in HMVEC medium and HTM medium under same imaging conditions.

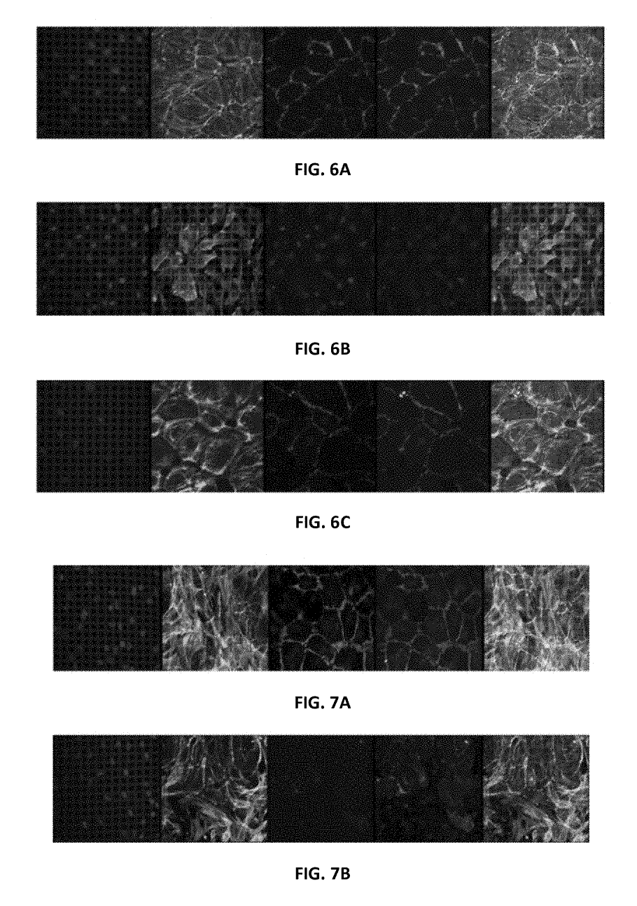

Furthermore, HMVECs grown on scaffolds in different media were stained with phalloidin to reveal cytoskeleton and immunostained with antibodies against PECAM-1 and fibulin-2. FIGS. 6A-6C show confocal images illustrating the effects of media (HMVEC medium, HTM medium, and HTM cell-conditioned medium, respectively) on HMVECs expression of HSC-markers after 7 days. From left to right are shown DAPI, F-actin, PECAM-1, fibulin-2, and merged. On day 7 the F-actin cytoskeleton of HMVECs in HMVEC medium appeared to be organized into short, unaligned stress fibers while PECAM-1 and fibulin-2 delineated cobblestone-like junctions and were also found diffused throughout cytoplasm. FIG. 6A. HTM medium induced cell retraction, in which some cells appeared slightly elongated with gaps between cells, exhibiting diffuse and nuclear expression of PECAM-1 and fibulin-2. FIG. 6B. Similarly to HMVEC medium, HTM cell-conditioned medium induced short, unaligned stress fibers and increased cortical F-actin staining, in which PECAM-1 and fibulin-2 outlined cell junctions and revealed larger polygonal cell shapes compared to cells cultured in HMVEC medium. FIG. 6C.

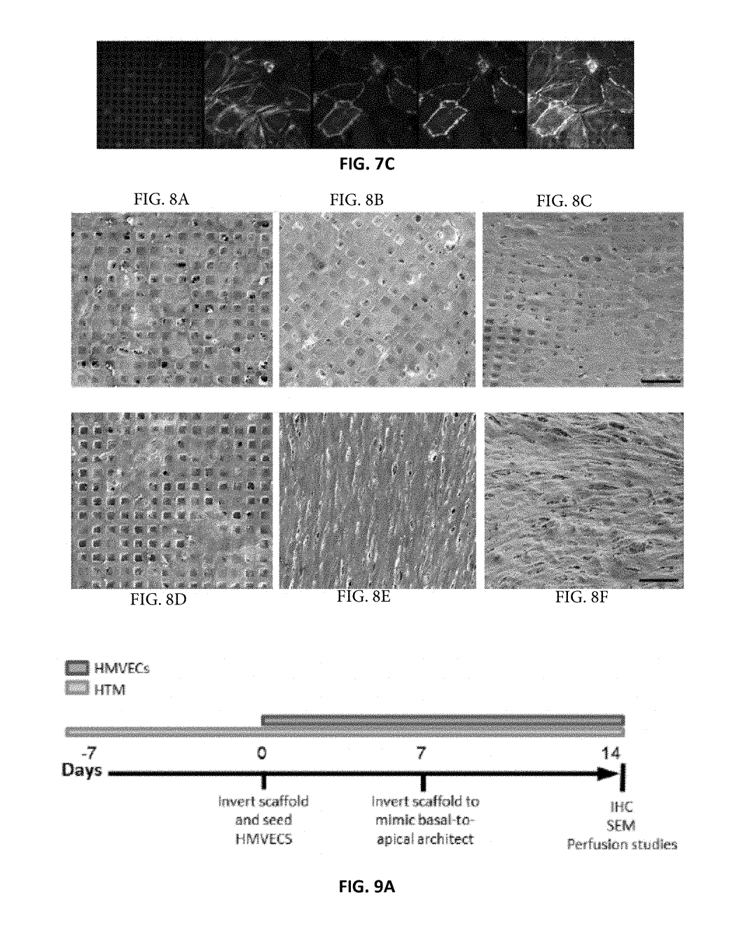

FIGS. 7A-7C show the effects of media (HMVEC medium, HTM medium, and HTM cell-conditioned medium, respectively) on HMVEC expression of HSC markers after 14 days. Panels from left to right show DAPI, F-actin, PECAM-1, fibulin-2, and merged. By day 14, HMVEC medium and HTM medium induced formation of disorganized longer stress fibers, some of which formed into denser microfilamentous fibers (FIGS. 7A-B) compared to the arrangement described by day 7 of culture. In addition, PECAM-1 and fibulin-2 expression, bordering the cell shape, was intensified in HMVEC medium and was greatly decreased in HTM medium. HTM cell-conditioned medium in contact with HMVECs for 14 days maintained cortical F-actin expression and preserved PECAM-1 and fibulin-2 at cell junctions (FIG. 7C). Therefore, all together, these results suggest that biochemical cues alone are not sufficient to effectively and consistently induce HSC-like differentiation of HMVECs into a HSC-like monolayer.

Differentiation of HMVECs to HSC-Like Cells by Co-Culture with HTM on SU-8 Scaffolds

FIGS. 8A-8F show scanning electron micrographs of HMVECs under co-culture with HTM for 7 (8A-8C) and 14 (8D-8F) days. From left to right, panels show HMVEC alone as a control, and top and tiled views of HMVECs cultured with HTM cells. HMVECs and HTM cells were co-cultured and cell morphology monitored via SEM on day 7 and day 14 of co-culture. After 7 days of HTM/HMVEC co-culture (FIGS. 8B-C) HMVECs appeared similar to mono-cultured cells (FIG. 8A). Co-cultured HMVEC cell layer was slightly thicker than mono-cultured controls. By day 14 of co-culture, striking differences were noted between HMVECs grown alone (FIG. 8D) and those co-cultured with HTM cells (FIGS. 8E-F). While mono-cultured HMVECs maintained polygonal morphology and thin cell layer appearance, HTM/HMVEC co-culture induced morphological changes on HMVECs which appeared elongated and the cell-layer was increasingly thicker.

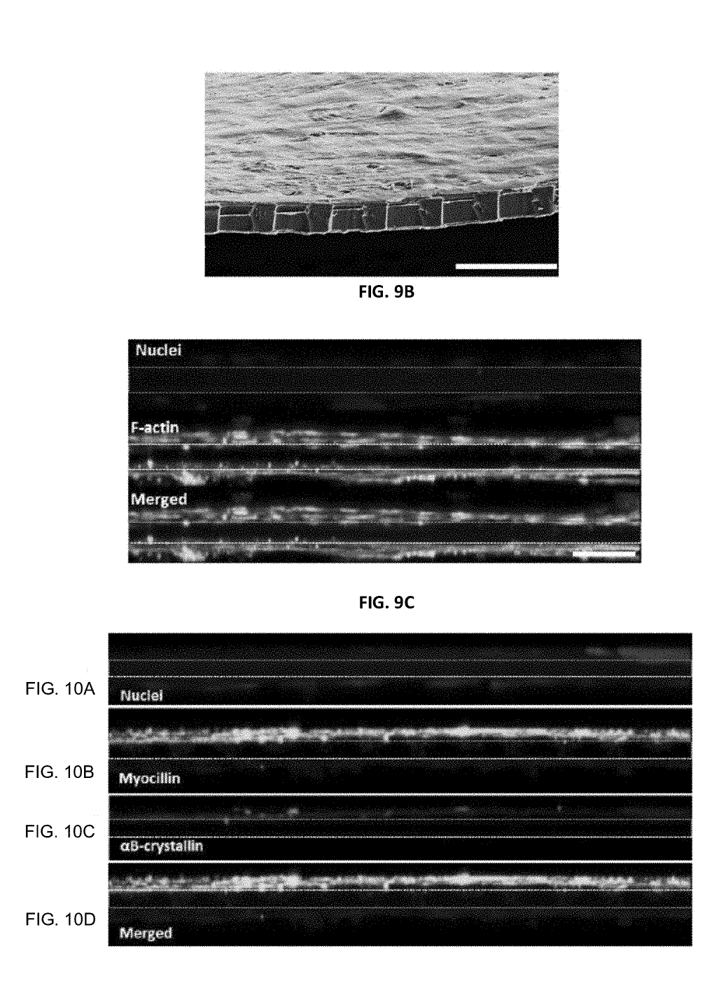

The HTM/HMVEC co-culture protocol was used for the following examples is diagrammatically illustrated in FIG. 9A. HTM cells were first cultured for 7 days on one side of an HA-coated SU8-scaffolds (from day −7 to day 0). On day 0 the scaffold is inverted and HMVECs were cultured on the opposite side to which HTM cells were cultured on. After allowing HMVEC cell attachment and initial growth, on day 7, constructs were again inverted to emulate the in vivo tissue architecture where HTM cells in the apical-to-basal direction, while HSCs are in the basal-to-apical direction. A scanning electron micrograph of co-cultured HTM/HMVEC cells on day 14 is shown in FIG. 9B. HTM/HMVEC constructs distinctively showed cell layers on each side of the scaffold. FIG. 9B. The co-cultured constructs were stained with phalloidin. Through confocal Z-stacks shown in FIG. 9C (from top to bottom, DAPI, F-actin, and merged, with the scaffold indicated by dotted lines) the side-view profile of the co-cultured samples were visualized demonstrating cell layers at both sides of the scaffolds. Cell processes that originated from each cell layer were observed running down the pores of the scaffolds in all of the samples. Some of these processes appeared to connect to each other, while others were shorter and did not reach the opposite cell layer.

To confirm that the co-culture of HTM cells with HMVECs was not inducing detrimental differentiation of the HTM cells, expression of HTM markers such as myocilin and αB-crystallin were analyzed on the co-cultured constructs. Through confocal imaging the expression of these two markers were visualized and by side-view rendering the location of myocilin and αB-crystallin limited to the HTM cell layer on the HTM/HMVEC constructs was confirmed. FIGS. 10A-10D show cross-sectional confocal imaging of DAPI, myocilin, αβ-crystalin, and merged, respectively, staining of HTM/HVEC co-cultures, with scaffolding indicated by dotted lines. These data demonstrate the feasibility of co-culturing HMVECs and HTM cells, and suggests that interactions, perhaps cell-cell contact and/or cell-ECM interaction, may induce morphological changes of HMVECs from cobblestone-like to an elongated denser cell-shape.

Comparison of 3D co-culture of HTM/HMVEC with HTM/HSC

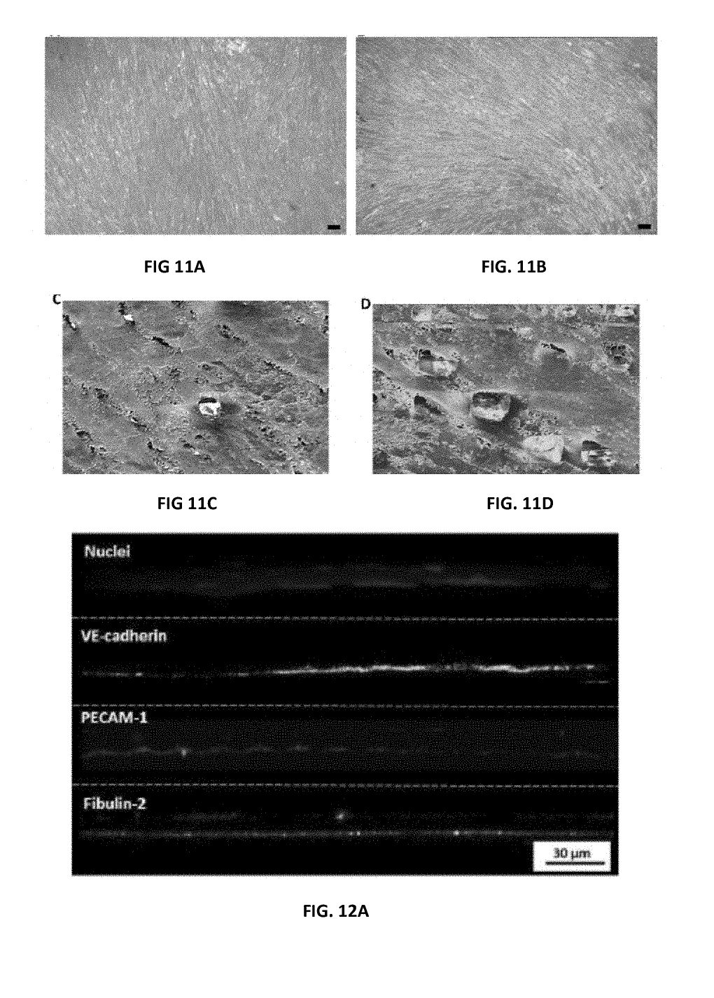

To investigate the feasibility of using the HTM/HMVEC construct as a platform to evaluate novel glaucoma therapeutics that target the conventional outflow tract, HTM/HMVEC co-culture was further characterized and directly compared to its HTM/HSC co-culture counterpart. FIGS. 11A-11D show HSC (11A and 11D) and HMVEC cells co-cultured with HTM cells for 14 days, with the cell layer shown in FIGS. 11A and 11B (scale bar=100 μm) and pore formation shown in FIG. 11C and FIG. 11D (scale bar=2 μm). The topography and cell morphology of these cells after 14 days of co-culture were compared side-by-side. HSC cells co-cultured with HTM cells grew to confluence, covering the SU-8 scaffold, and presented elongated, spindle-like shape. FIG. 11A. Similarly, HMVECs co-cultured with HTM cells also covered the scaffold exhibited elongated shape. FIG. 11B. Vacuole and pore formation by HSC cells may be a mechanism for regulation of outflow resistance. Therefore, vacuole/pore formation was attempted by perfusing the HSC and/or HMVEC cell layer in co-cultured constructs in a basal-to-apical direction. SEM analysis demonstrated that both, HSC and HMVEC layers form vacuoles/pores after perfusion. FIGS. 11C-D. Vacuoles observed on the HSC layer had dimensions of 4.25±1.05 μm, 4.51±0.7 (length, width) (N=15, over 2 experiments) while HMVECs presented vacuoles of 6.25±0.53 μm in length by 4.56±1.1 μm in width (N=12 over 3 experiments). All vacuoles observed had small orifices at the surface which might be due to the electron beam that caused the opening to widen during imaging.

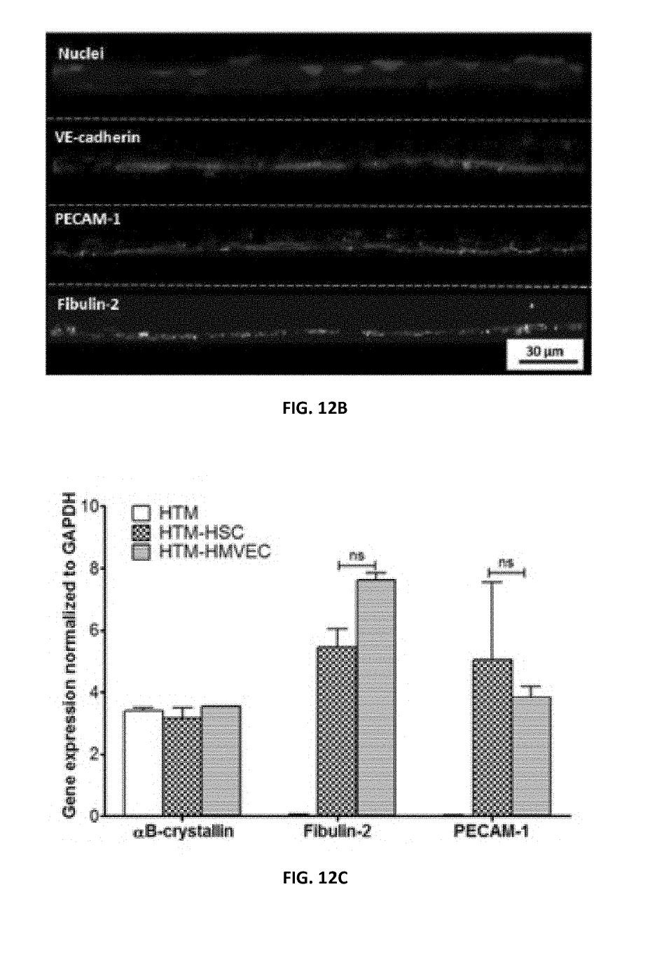

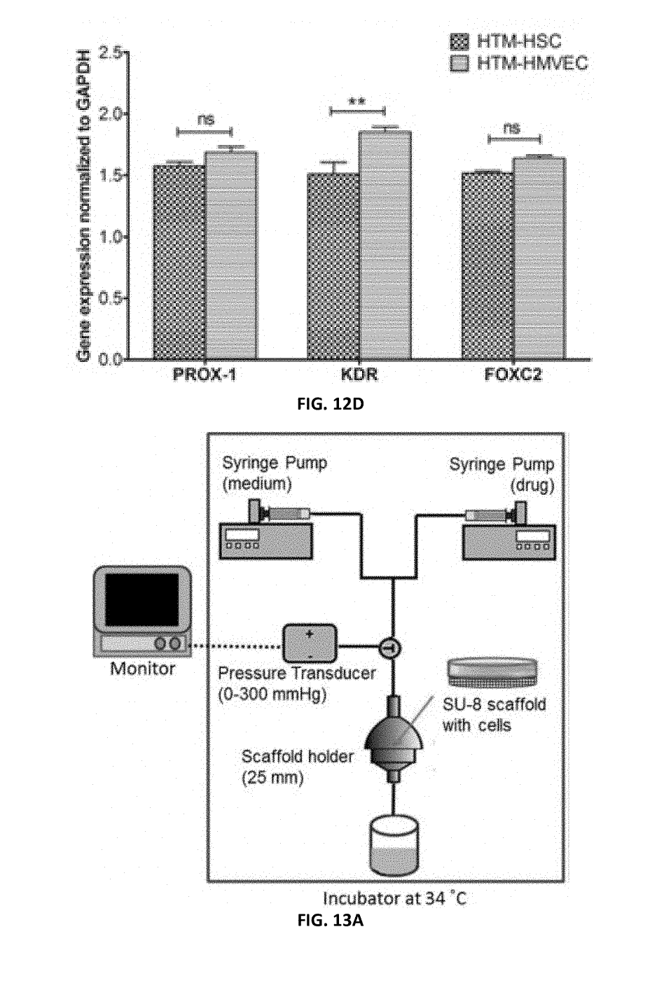

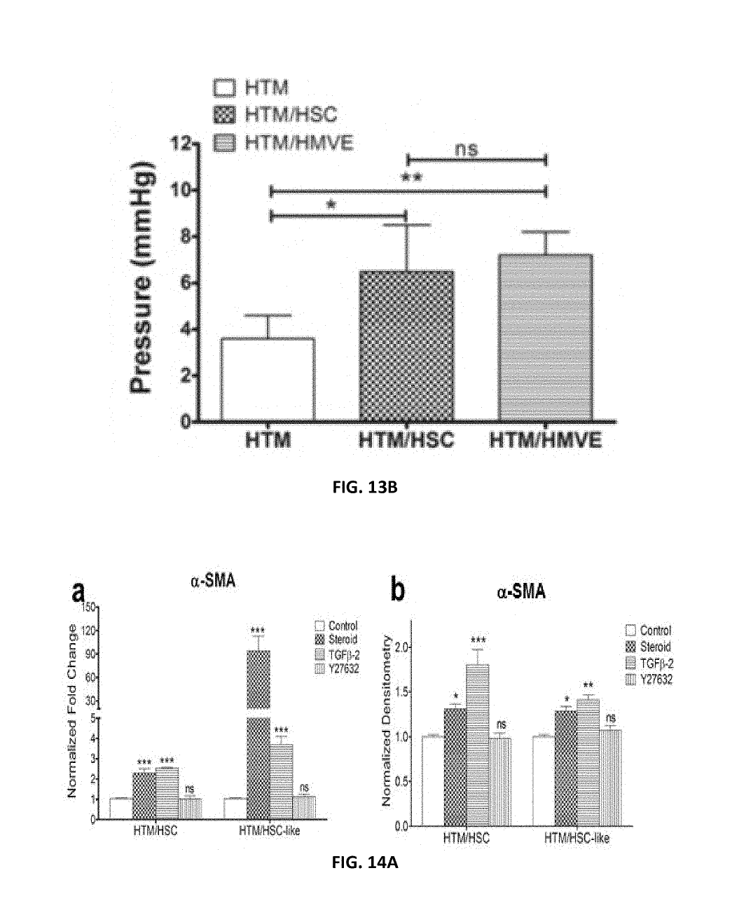

The expression of VE-cadherin, PECAM-1 and fibulin-2 between co-cultured HTM/HSC and HTM/HMVEC constructs was also compared. Side-view rendering of confocal z-stack images allowed visualization of the expression of these markers. FIGS. 12A (HSC/HTM) and 12B (HMVEC/HTM) show 3D co-culture of HTM/HMVEC constructs (indicated cell types co-cultured for 14 days on HA-coated SU-8 scaffold), and their maintenance of HSC-marker expression, via cross-sectional confocal imaging (from top to bottom, DAPI, VE-cadherin, PECAM-1, and fibulin-2). Both constructs expressed VE-cadherin, PECAM-1 and fibulin-2 at the HSC (FIG. 12A) and HMVEC (FIG. 12B) cell layer. PECAM-1 and fibulin-2 expression appeared to be slightly higher on the HTM/HMVEC samples, while VE-cadherin expression appeared to be intensified in HTM/HSC constructs.

To determine whether these qualitative differences were significant and to confirm the presence of the HTM layer in the co-cultured samples, we performed qPCR analysis of gene expression of HTM-marker of αB-crystallin, HSC markers of PECAM-1 and fibulin-2 and house-keeping gene of GAPDH (Table 2) in HTM alone, HTM/HSC and HTM/HMVEC 3D constructs. Gene expression of HTM and key HSC markers in 3D co-cultured HTM/HMVEC constructs is shown graphically in FIGS. 12C and 12D. qPCR analysis confirmed that 3D co-culture HTM/HMVEC constructs maintained HTM- and HSC-marker expression in comparison to HTM alone and HTM/HSC co-culture. Samples were analyzed using two-way ANOVA followed by Bonferroni post-tests. Asterisks indicate significant differences (** p<0.01). The αB-crystallin gene was expressed in all samples, corroborating the presence of the HTM layer in co-cultured constructs. FIG. 12C. PECAM-1 and fibulin-2 genes were not expressed in 3D HTM constructs. On the contrary, as expected, HTM/HSC and HTM/HMVEC 3D constructs indeed expressed PECAM-1 and fibulin-2 genes. FIG. 12C. PROX-1, KDR, and FOXC2 were also expressed in HTM-HSC constructs and HTM-HMVEC constructs. FIG. 12D.

Additionally, expression of ECM proteins such as collagen IV, fibronectin and laminin were confirmed in the HTM/HMVEC co-cultured constructs. Gene expression of these ECM proteins in HTM/HSC and HTM/HMVEC samples were further compared. Results showed that HTM/HMVEC constructs express less collagen IV (N=3, P<0.001) and fibronectin (N=3, P<0.05) than HTM/HSC co-cultures.

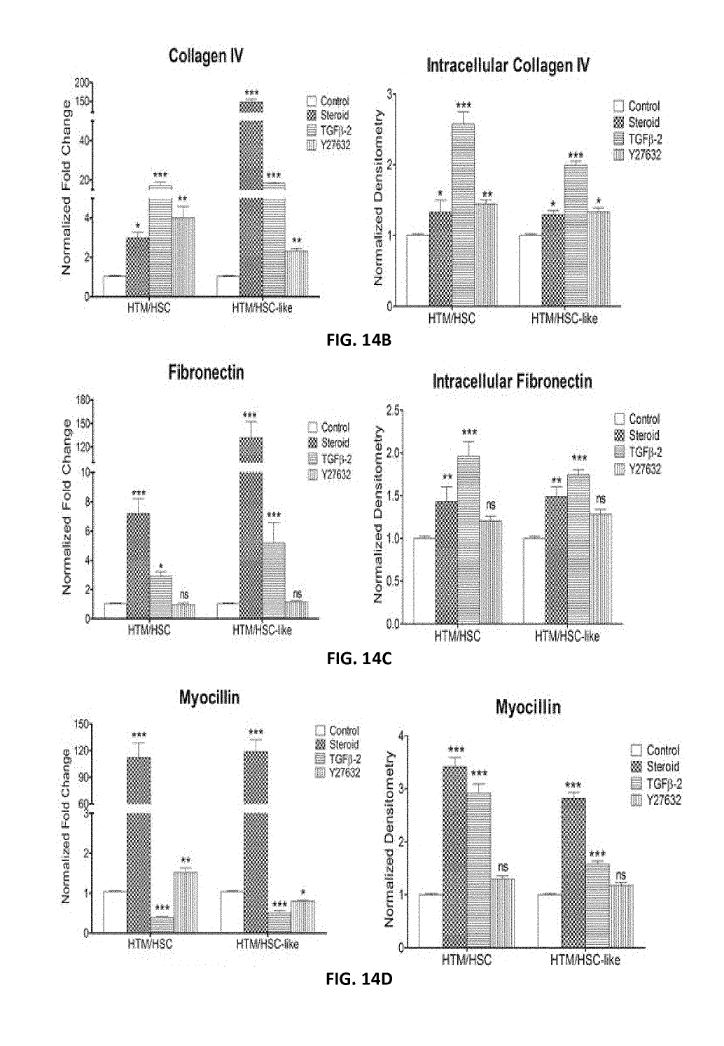

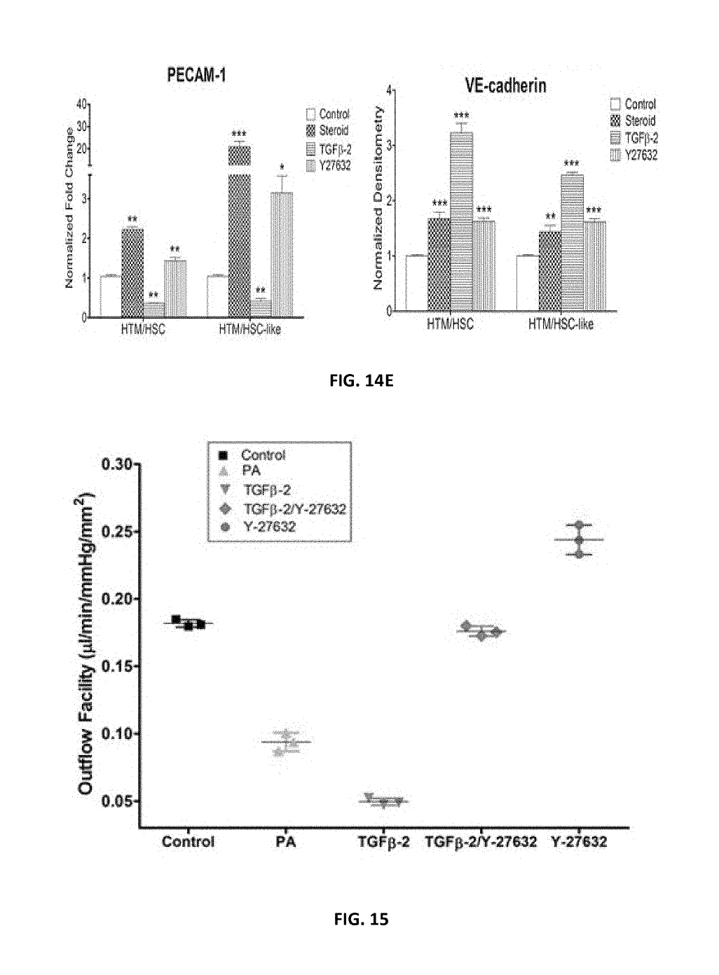

The co-cultured HTM-HMVEC outflow system also responds similar to the HTM-HSC system to agents that either decrease (e.g., steroid, TGF-β) or increase (e.g., Y27632) outflow facility. FIGS. 14A-E show gene (left panels, qPCR) and protein (right, western blots) expression patterns of co-cultured HTM/HVEC constructs. HTM-HMVEC and HTM-HSC constructs were contacted with pharmacologic agents that regulate outflow and expression levels of various genes and proteins (α-SMA, collagen IV, fibronectin, myocillin) measured by qPCR and western blotting. The effect agents that decrease outflow activity had on expression levels in HTM/HSC constructs were directionally similar to the effects they had on HTM/HMVEC constructs, as were the effects of agents that increase outflow activity. * p<0.05. ** p<0.01. *** p<0.001.

Porous scaffolds with HTM cells cultured respond in a predictable manner to treatment with pharmacological agents that are known to modulate outflow activity. FIG. 15 is a graphical representation of the effects of agents that inhibit (prednisone acetate (PA), TGFβ) or stimulate (Y27632) outflow facility. As shown, agents known to inhibit or potentiate outflow facility inhibit or potentiate outflow facility of porous scaffolds bearing cultured HTM cells, respectively. Given the consistent activity and responsiveness of HTM/HSVEC and HTM/HSC constructs in accordance with aspects of the present disclosure and the examples presented herein, a skilled artisan would appreciate that effects of agents that promote or oppose IOP pathology would have cytological, biochemical, morphological, or functional effects of HTM/HSVEC co-cultured porous constructs in accordance with aspects of the present disclosure that are consistent with their effects on physiological functioning of the conventional outflow tract, and that effects of treatments of constructs in accordance with aspects of the present disclosure would be predictive of pathological, therapeutic, or prophylactic effect clinically.

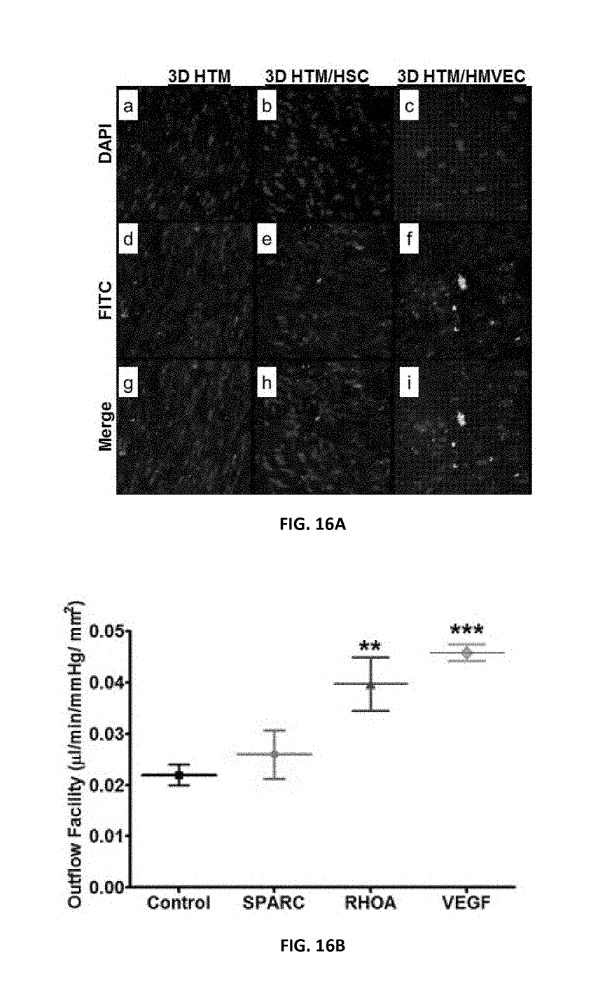

Conventionally, transfection of TM and SC cells is also difficult. Cellular transfection of cells grown on a porous scaffold in accordance with aspects of the present disclosure is an improvement over traditional methods permitting more robust transfection for the manipulation of gene expression. To date, transfection of HTM cells with plasmids or siRNA has been challenging and requires the use of electroporation of cells in suspension. The combination of culturing HTM and SC cells on an SU-8 scaffold in accordance with the aspects of the present disclosure and using Targefect agents (Targeting Systems), which are specifically designed for the transfection of a macrophage-like cell line allows for almost 100% of the cells to internalize fluorescent siRNAs. The Ras homolog (Rho) and RHO-associated, coiled-coil containing protein kinase (ROCK) pathway were targeted with si-RhoA mimicking the pharmacologically decreased actin stress-fiber formation and alters architecture of the SC-monolayer. Confocal images showed that 3D HTM/HMVEC constructs can be transfected by siRNA using Targefect RAW reagent similar to 3D HTM and 3D HTM/HSC constructs. FITC-labeled control siRNA was used as the reporter. FIG. 16 shows confocal micrographs of HTM cells (left panels) or HTM cells co-cultured with HSC (middle panels) or HMVEC cells (right panels) on 3D scaffolds and transfected with FITC-labeled siRNA using Targefect RAW transfection agent (from top to bottom, DAPI, FITC, and merged). These results demonstrate that cells cultured in accordance with aspects of the present disclosure are highly amenable to transfection.

Long-term, properly targeted transgene expression in the trabecular meshwork and Schlemm's canal cells constituting the conventional outflow tract has not been achieved by conventional methods. As shown in FIG. 16B, when HTM-only constructs were transfected with siRNA species designed to knock down expression of RhoA and VEGF, which are known to increase IOP, outflow facility of such constructs was increased. FIG. 16B shows outflow facility in control constructs and constructs treated with siRNA that knocks down expression of SPARC, RhoA siRNA, or VEGF (** p<0.01; *** p<0.001). Uptake of siRNA by porous scaffold constructs and alterations in function in accordance with aspects of the present disclosure demonstrates that transfection for the purpose of gene regulation is also possible with such constructs.

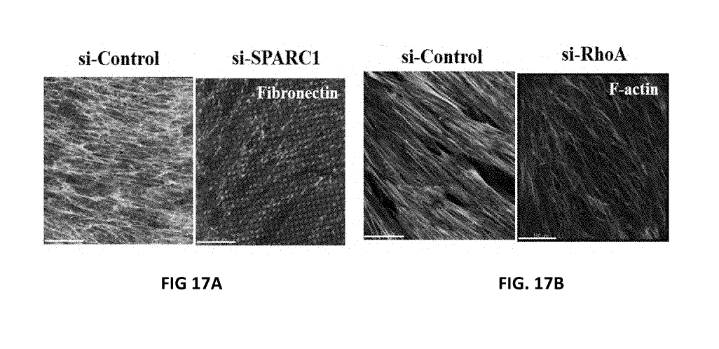

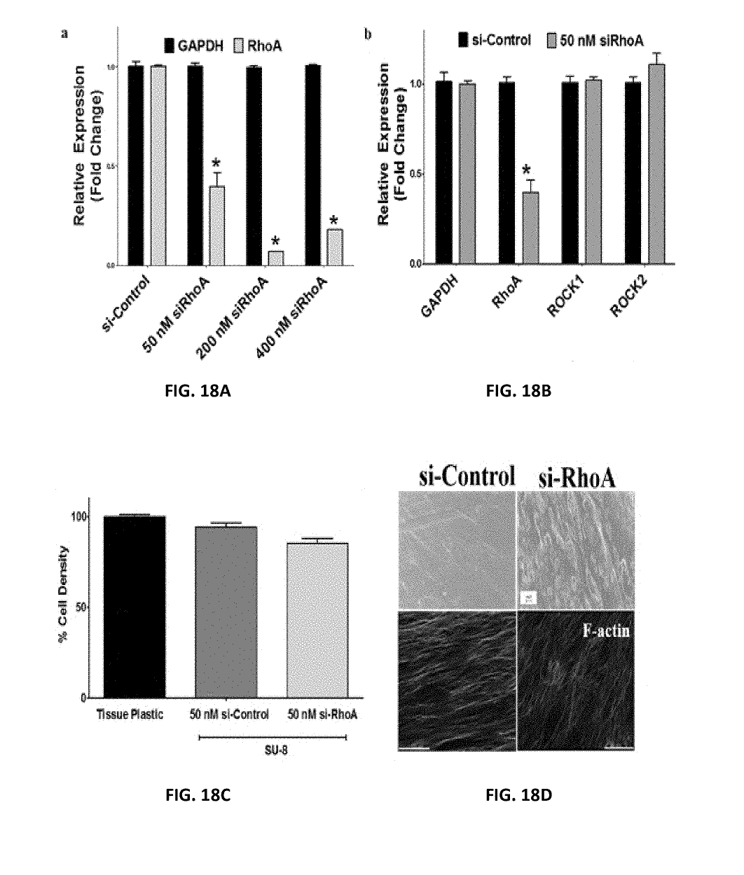

Further examples are shown in FIGS. 17A-17B and FIGS. 18A-18D. HTM or HSC cells were seeded on Extracel-coated 12 μm-pore SU-8 scaffolds and cultured for 10 days (HSC) or 14 days (HTM) to confluence. Upon reaching confluence, HSC cells were fixed for immunohistochemistry analysis or scanning electron microscopy (SEM) or used for drug treatment and/or perfusion studies as described in subsequent sections.

After 11 days in culture (˜70% confluence), HTM cells or HSC cells were transfected with FITC-siRNA as a reporter siRNA, 200 nM si-β-actin, 25, 50, 200, or 400 nM si-Rho, 50, 200, or 400 nM si-SPARC, or scrambled siRNA (si-Control) (Santa Cruz Biotechnology) according to the transfection factor manufacturer's (Targeting System) protocol. To 0.6 ml of high glucose DMEM (Life Technologies), 12 μl of Targefect reagent was added along with the appropriate siRNA. The tube was flicked 12 times. Subsequently, 25 μl of the Virofect enhancer was added and the tube was flicked 12 times and incubated for 25 min at 37° C. 1.2 ml of 10% FBS/high glucose DMEM was then added to the tube. 225 μl of the transfection complex was added to each cell scaffold construct. Following an overnight incubation, the transfection complex was removed and fresh 10% FBS/DMEM was added to the cells in each well.

The 3D HTM or HSC cell-scaffold constructs were washed with 1× phosphate buffered saline (PBS; Life Technologies, Carlsbad, Calif.) three times and fixed in 4% paraformaldehyde (Sigma Aldrich) in PBS for 15 min After fixation, the construct was washed three times in 1×PBS, permeabilized in 1% Triton X-100 (Sigma Aldrich) in 1×PBS, and blocked with 5% FBS in PBS for 1 hour. For immunocytochemistry, 3D HTM samples were incubated with the primary antibody against fibronectin, washed five times in 1×PBS, followed by incubation with goat anti-rabbit Alexa Fluor 488 ( 1/100) (Life Technologies, Carlsbad, Calif.). For cytoskeletal staining, HTM or HSC samples were incubated overnight at 4° C. with phalloidin 488 diluted in 1×PBS. Finally, samples were washed three times with 1×PBS and mounted onto glass slides with Prolong Gold Antifade Reagent (Life Technologies). Laser scanning confocal microscopy was performed using a Leica SP5 confocal microscope (Leica Microsystems, Mannheim, Germany) and images were acquired at 40× magnification with an oil-immersion objective. Confocal images were processed using Imaris software 7.7 (Bitplane Inc, South Windsor, Conn.), and all images within a given experiment were captured using the same laser intensity and gain settings in order to be able to compare intensities across samples.

PCR probes and primers were generated using Integrated DNA Technologies probe/primer design software (http://www.idtdna.com/pages/products/geneexpression/primetime-qper-assays-and-primers). Total RNA was extracted from confluent HSC cultured on tissue culture plastic, Extracel-coated tissue culture plastic plates, or Extracel-coated SU-8 scaffolds cells using RNeasy Mini Kits as per the manufacturer's instructions (Qiagen, Hilden, Germany). RNA was quantified using a NanoDrop ND1000 Spectrophotometer (Thermo Scientific, Wilmington, Del.). qRT-PCR was carried out as directed using TaqMan RNA-to-C_{T}™ 1-Step Kit (Applied Biosystems, Carlsbad, Calif.) using 100 ng of RNA for each samples. qRT-PCR was performed on an AB StepOnePlus Real Time PCR system (Life Technologies, Carlsbad, Calif.) using an RT step (48° C. for 15 min) followed by an enzyme activation step (95° C. for 10 min), then 40 cycles of 15 s denaturation at 95° and 1 min anneal/extend at 60° C. Relative quantitation data analysis was performed using the comparative quantification method, ΔΔCt, with GAPDH as the endogenous reference. Three replicates were performed and the average values are presented as mean±SD with p<0.05 considered significant.

After 14 days in culture, cell viability was measured using a Vybrant MTT Cell Proliferation Assay Kit (Life Technologies). All assays were performed using improved minimal essential media (IMEM; Cellgro) as it does not contain phenol red, which may compromise assay results. As per the manufacturer's protocol, 10 μL of 12 mM MTT stock solution in 100 μL of IMEM was added to each cell-scaffold construct. Samples were then incubated for 4 hr at 37° C. After the 4 hr incubation, cells were solubilized by addition of 100 μL of SDS-HCl solution to release the formazan product. Samples were incubated overnight and the absorbance at 570 nm was measured using a Tecan infinite M200 plate reader (Tecan Inc, Männedorf, Switzerland). Viable cell density was normalized by the absorbance at 570 nm of cells grown on tissue culture plastic.

siRNA gene silencing was used to downregulate genes that play roles in aqueous outflow regulation and function, RhoA and SPARC. RhoA signaling is responsible for cytoskeletal dynamics in the HTM or HSC in response to aqueous humor outflow, mediating stress fiber formation. SPARC mediates the increase in ECM deposition associated with a glaucomatous state. Sensitivity of the engineered HTM or HSC constructs to siRNA treatment was evaluated. Dose responsiveness of inhibition of RhoA expression in response to siRhoA in 3D HTM constructs was confirmed, with 50 nM siRNA causing the most significant RhoA downregulation. ROCK1 and ROCK2 are two isoforms of ROCK, which are activated by RhoA binding. There was no significant was down-regulation of the RhoA downstream effectors, ROCK1 and ROCK2, by siRhoA, just as expected since RhoA regulates ROCK1 and ROCK2 post-transcriptionally. Additionally, β-actin expression was also unaffected by transfection with 50 nM si-RhoA

The effect of si-SPARC1 treatment in 3D HTM constructs was evaluated. The 3D HTM constructs exhibited significant SPARC1 down-regulation for all the doses evaluated, ranging from 50 nM to 400 nM si-SPARC1. SPARC1 target genes, collagen I and fibronectin, were significantly down-regulated following transfection with 50 nM si-SPARC1, as expected. Gene expression of GAPDH was not affected by either siRhoA or siSPARC1, demonstrating selectivity of both siRNAs. Since maximum inhibition of RhoA or SPARC1 was observed at 50 nM, 50 nM was selected as the dose for further assessment of the effects of siRNAs on the 3D HTM constructs.

Immunocytochemistry confirmed decreased fibronectin expression in 3D HTM constructs after transfection with 50 nM si-SPARC1 as shown in FIG. 17A, while phalloidin staining of the F-actin cytoskeleton confirmed F-actin downregulation following treatment with 50 nM si-RhoA, as shown in FIG. 17B.

The effects of si-RhoA or si-SPARC1 on HTM viable cell density as a measure of cytotoxicity were also measured. Three days following transfection with 50 nM si-RhoA or Si-SPARC1, there was no significant difference in the viable cell density between the si-Control and si-RhoA or Si-SPARC1 treated samples, indicating that treatment with si-RhoA or si-SPARC1 has no adverse effects on HTM growth or viability. The nearly two-fold increase in cell density in accordance with aspects of the present disclosure when compared to conventional tissue culture plastic control is not surprising as the SU-8 scaffold disclosed herein facilitated 3D culture of HTM cells, which results in the formation of multiple (3) layers of cells as compared to the single layer in a conventional 2D culture. Together, these experiments demonstrate for the first time that 3D culture of HTM cells on well-defined porous SU-8 scaffolds fosters high efficiency gene transfer. Plasmid gene transfer is also effective (not shown).

In a similar fashion to the biomimetic HTM, effects of si-RhoA treatment on the engineered 3D HSC constructs was also tested. qRT-PCR analysis confirmed a dose-dependent response of the HSC monolayer to varying concentrations of si-RhoA, as shown in FIG. 18A. There was no significant down-regulation of the RhoA downstream effectors, ROCK 1 or ROCK2 after transfection with 50 nM si-RhoA as observed in the HTM, as shown in FIG. 18B. GAPDH expression was not affected by si-RhoA, indicating selective targeting of RhoA.

The effect of si-RhoA on HSC viable cell density was also tested. Three days following transfection with 50 nM si-RhoA, there was no significant difference in cell viable cell density between si-Control or si-RhoA samples, as shown in FIG. 18C, demonstrating that treatment with si-RhoA had no adverse effect on HSC growth or viability. Scanning electron microscopy (FIG. 18D, top panels) confirmed that a continuous HSC cell layer was present for both si-Control and si-RhoA treated samples. The distinct surface morphology observed in the si-RhoA sample can be explained by the decrease in stress on actin regulation as a result of si-RhoA treatment, as revealed by F-actin staining in the bottom panels of FIG. 18D. According to conventional methodology and systems, HSC cells transfection has only been reported for cells in suspension via Nucleofection using the Lonza Nucleofector™ However, if transfected cells are replated in a traditional 2D culture system, scenescence, loss of key in vivo characteristics, and eventual loss of the transgene may occur, reducing the utility of that approach for screening gene therapy treatments. In accordance with aspects of the methods and systems disclosed herein, well-defined, microporous SU-8 scaffolds may facilitate an increase in the surface area for high efficiency gene transfer and together with the Extracel coating, allow for basolateral access to media for maintenance of their characteristic in vivo characteristics, advantageously improving maintenance of a transgene for longer period of time.

Altogether, these data suggests the HTM/HMVEC and HTM/HSC co-culture have striking phenotypical similarities, posing a novel tool to study the differentiation of blood endothelial cells to HSC-like and a higher-throughput platform to investigate novel intraocular pressure lowering agents/targets. Any of the various constructs disclosed herein—trabecular meshwork cells, Schlemm's canal cells, Schlemm's canal cell-like cells, microvascular cells, or any combination of two or more of the foregoing, may be grown or maintained on a scaffold in accordance with aspects of the present disclosure and be transfected with agents such as siRNA, plasmid DNA, antisense RNA, or other agents that may directly regulate gene expression or activity. Skilled artisans would appreciate that any of various widely known methods of transfection may be used to transfect cells in accordance with aspects of the present disclosure, such as chemically mediated methods, particle-mediated methods, viral vector-mediated gene transfer or other viral transduction techniques, nucleofection, or other methods, which could be readily adapted to use with systems, methods, or constructs in accordance with aspects of the present disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to possessing only those one or more steps or elements. Likewise, a step of a method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description of the present disclosure has been presented for purposes of illustration and description, but is not intended to be exhaustive or limited in the form disclosed. Many modifications and variations will be apparent to those of ordinary skill in the art without departing from the scope and spirit of the disclosure. The embodiments were chosen and described in order to best explain the principles of one or more aspects of the disclosure and the practical application, and to enable others of ordinary skill in the art to understand one or more aspects of the disclosure for various embodiments with various modifications as are suited to the particular use contemplated.

What is claimed is:

1. A system for modeling a conventional outflow tract, comprising:
a porous scaffold comprising a first surface and a second surface and the first surface is on a side opposite the second surface;
a plurality of trabecular meshwork cells attached to the first surface and extending into a plurality of pores in the porous scaffold; and
a plurality of second cells attached to the second surface and extending into the plurality of pores, wherein the second cells comprise microvascular endothelial cells that were co-cultured on the scaffold with the plurality of trabecular meshwork cells for a period of time to form Schlemm's canal cell-like cells, Schlemm's canal cells, or both.

2. The system of claim 1, wherein the porous scaffold comprises a micropatterned substrate.

3. The system of claim 2, wherein the micropatterned substrate comprises a photoresist, a thermoplastic polymer, or a thermoset polymer.

4. The system of claim 1, wherein the porous scaffold comprises a coating.

5. The system of claim 1, wherein the porous scaffold comprises a pore width of between 200 nm and 1 μm, between 1 μm and 5 μm, between 5 μm and 10 μm, between 10 μm and 15 μm, between 15 μm and 20 μm, or between 7 μm and 15 μm.

6. The system of claim 1, wherein the plurality of trabecular meshwork cells, the plurality of second cells, or both, are transfected or are contacted with a known or suspected medicament or a compound known or suspected to impair, enhance, ameliorate, or improve conventional outflow tract function.

7. A method for screening employing the system of claim 1, comprising:
a) obtaining the porous scaffold having the plurality of trabecular meshwork cells and the plurality of second cells attached thereto, the plurality of trabecular meshwork cells having been formed by seeding and growing the plurality of trabecular meshwork cells on the first surface, the plurality of second cells having been formed by seeding and growing the plurality of second cells on the second surface, wherein the second cells comprise microvascular endothelial cells that were co-cultured with the plurality of trabecular meshwork cells on the first surface for a period of time at least until the plurality of microvascular endothelial cells were transformed into a plurality of Schlemm's canal cell-like cells, a plurality of Schlemm's canal cells, or both;
b) contacting the plurality of trabecular meshwork cells, the plurality of second cells, or both, with a known or suspected medicament or a compound known or suspected to impair, enhance, ameliorate, or improve conventional outflow tract function, or transfecting the plurality of trabecular meshwork cells, the plurality of second cells, or both;
c) perfusing fluid through the plurality of trabecular meshwork cells and the plurality of second cells; and
d) measuring a transmembrane pressure, a flow rate, a through-flow, a resistance to flow, a hydraulic conductivity, an electrical conductivity, a vacuole dynamics, a pore formation, a biomarker analysis of a perfusate, or an outflow facility of the plurality of trabecular meshwork cells and the second cells.

8. The method of claim 7, wherein the seeding and growing of the plurality of trabecular meshwork cells comprises seeding at an initial density of trabecular meshwork cells of greater than 10,000 cells/cm².

9. The method of claim 7, wherein the seeding and growing of the plurality of second cells comprises seeding at an initial density of second cells of greater than 10,000 cells/cm².

10. A method of making the system for modeling a conventional outflow tract of claim 1, comprising:
obtaining a porous scaffold wherein the porous scaffold comprises a first surface and a second surface and the first surface is on a side opposite the second surface;
seeding and growing a plurality of trabecular meshwork cells on the first surface; and
seeding and growing a plurality of second cells on the second surface wherein the second cells comprise Schlemm's canal cells or are microvascular endothelial cells and co-culturing the plurality of trabecular meshwork cells on the first surface for a period of time at least until the plurality of microvascular endothelial cells are transformed into a plurality of Schlemm's canal cell-like cells.

11. A system for modeling a conventional outflow tract, comprising:
a porous scaffold comprising a first surface and a second surface and the first surface is on a side opposite the second surface;
a plurality of first cells attached to the first surface and extending into a plurality of pores in the porous scaffold, wherein the first cells comprise trabecular meshwork cells, stem cells that can differentiate into trabecular meshwork cells, precursor trabecular meshwork cells, or any combination of two or more of the foregoing; and
a plurality of second cells attached to the second surface and extending into the plurality of pores, wherein the second cells comprise microvascular endothelial cells that were co-cultured on the scaffold with the plurality of first cells for a period of time to form Schlemm's canal cell-like cells, stem cells that can differentiate into vascular endothelial cells that were co-cultured on the scaffold with the plurality of first cells for a period of time to form Schlemm's canal cell-like cells, precursor vascular endothelial cells that were co-cultured on the scaffold with the plurality of first cells for a period of time to form Schlemm's canal cell-like cells, stem cells that can differentiate into Schlemm's canal cells that were co-cultured on the scaffold with the plurality of first cells for a period of time to form Schlemm's canal cell-like cells, Schlemm's canal cells, or any combination of two or more of the foregoing.

12. The system of claim 11, wherein the porous scaffold comprises a micropatterned substrate.

13. The system of claim 12, wherein the micropatterned substrate comprises a photoresist, a thermoplastic polymer, or a thermoset polymer.

14. The system of claim 11, wherein the porous scaffold comprises a coating.

15. The system of claim 11, wherein the porous scaffold comprises a pore width of between 200 nm and 1 μm, between 1 μm and 5 μm, between 5 μm and 10 μm, between 10 μm and 15 μm, between 15 μm and 20 μm, or between 7 μm and 15 μm.

16. The system of claim 11, wherein the plurality of trabecular meshwork cells, the plurality of second cells, or both, are transfected or are contacted with a known or suspected medicament or a compound known or suspected to impair, enhance, ameliorate, or improve conventional outflow tract function.

17. A method for screening employing the system of claim 11, comprising:
a) obtaining the porous scaffold having the plurality of first cells and the plurality of second cells attached thereto;
b) contacting the plurality of first cells, the plurality of second cells, or both, with a known or suspected medicament or a compound known or suspected to impair, enhance, ameliorate, or improve conventional outflow tract function, or transfecting the plurality of first cells, the plurality of second cells, or both;
c) perfusing fluid through the plurality of first cells and the plurality of second cells; and
d) measuring a transmembrane pressure, a flow rate, a through-flow, a resistance to flow, a hydraulic conductivity, an electrical conductivity, a vacuole dynamics, a pore formation, a biomarker analysis of a perfusate, or an outflow facility of the plurality of first cells and the plurality of second cells.

18. The method of claim 17, wherein the plurality of first cells is seeded and grown on the first surface, and the seeding comprises seeding at an initial density of first cells of greater than 10,000 cells/cm².

19. The method of claim 17, wherein the plurality of second cells is seeded and grown on the second surface, and the seeding comprises seeding at an initial density of second cells of greater than 10,000 cells/cm².

20. A method of making the system for modeling a conventional outflow tract of claim 11, comprising:
obtaining a porous scaffold wherein the porous scaffold comprises a first surface and a second surface and the first surface is on a side opposite the second surface;
seeding and growing a plurality of first cells on the first surface, wherein the first cells comprise trabecular meshwork cells, stem cells that can differentiate into trabecular meshwork cells, precursor trabecular meshwork cells, or any combination of two or more of the foregoing; and
seeding and growing a plurality of second cells on the second surface wherein the second cells comprise microvascular endothelial cells that form Schlemm's canal cell-like cells when co-cultured on the scaffold with the plurality of first cells for a period of time, stem cells that can differentiate into vascular endothelial cells that form Schlemm's canal cell-like cells when co-cultured on the scaffold with the plurality of first cells for a period of time, precursor vascular endothelial cells that form Schlemm's canal cell-like cells when co-cultured on the scaffold with the plurality of first cells for a period of time to form Schlemm's canal cell-like cells, stem cells that can be differentiated to Schlemm's canal cells that were co-cultured on the scaffold with the plurality of first cells for a period of time to form Schlemm's canal cell-like cells, Schlemm's canal cells, or any combination of two or more of the foregoing.


SEQUENCE LISTING (14 sequences)

SEQ ID NO: 1 - DNA, 21 bp, Homo sapiens
caatcacatctcccaacacct

SEQ ID NO: 2 - DNA, 21 bp, Homo sapiens
ctggtttgacactggactctc

SEQ ID NO: 3 - DNA, 20 bp, Homo sapiens
attgctctggtcacttctcc

SEQ ID NO: 4 - DNA, 16 bp, Homo sapiens
caggcccattgttccc

SEQ ID NO: 5 - DNA, 19 bp, Homo sapiens
ccaggcactcgtcattgtc

SEQ ID NO: 6 - DNA, 22 bp, Homo sapiens
ccaactctgtccattctatccc

SEQ ID NO: 7 - DNA, 19 bp, Homo sapiens
cctttgtgccattgcatcc

SEQ ID NO: 8 - DNA, 21 bp, Homo sapiens
gaacaaaagggacaagaggac

SEQ ID NO: 9 - DNA, 21 bp, Homo sapiens
gtccttgtgtcctgatcgttg

SEQ ID NO: 10 - DNA, 21 bp, Homo sapiens
aggctggatgatggtagattg

SEQ ID NO: 11 - DNA, 20 bp, Homo sapiens
agtgctctcctgttgtgttc

SEQ ID NO: 12 - DNA, 17 bp, Homo sapiens
gaatgtgtgccctgcga

SEQ ID NO: 13 - DNA, 22 bp, Homo sapiens
tgtagttgaggtcaatgaaggg

SEQ ID NO: 14 - DNA, 19 bp, Homo sapiens
acatcgctcagacaccatg